(19) United States
(12) Patent Application Publication
(10) Pub. No.: US 2019/0211364 A1
(43) Pub. Date: Jul. 11, 2019

(54) COMPOSITIONS AND METHODS FOR PRODUCING CITRUS TERPENOIDS

(71) Applicant: INTERNATIONAL FLAVORS & FRAGRANCES INC., New York, NY (US)

(72) Inventors: Ingo De Abreu, Princeton, NJ (US); Mauricio Poulsen, (SG)

(21) Appl. No.: 16/310,910

(22) PCT Filed: Jun. 30, 2017
(86) PCT No.: PCT/US17/40224

§ 371 Date: Dec. 18, 2018

Related U.S. Application Data
(60) Provisional application No. 62/357,618, filed on Jul. 1, 2016.

(51) Int. Cl. C12P 5/00; C12N 9/88; C12N 9/02

(52) U.S. Cl. CPC C12P 5/007; C12Y 106/02004; C12N 9/0042; C12N 9/88

(57) ABSTRACT

Compositions and methods for synthesizing a citrus terpenoid using a recombinant host cell that produces isopentenyl pyrophosphate and dimethylallyl pyrophosphate, and expresses one or more enzymes that convert the IPP and DMAPP to a citrus terpenoid are described.

### INTRODUCTION

This patent application claims the benefit of priority from provisional U.S. Patent Application Ser. No. 62/357,618, filed Jul. 1, 2016, the contents of which are incorporated herein by reference in their entirety.

### BACKGROUND

The term “terpene” refers to a class of compounds derived from isoprene, which has the molecular formula C₅H₈. The basic molecular formula for terpenes includes multiples of C₅H₈, that is, (C₅H₈)ₙ where n is the number of linked isoprene units. The isoprene units may be linked together to form linear chains, or they may be arranged to form rings. Terpenes are classified by the number of terpene units in the molecule. Whereas monoterpenes are composed of two condensed basic units of isopentenyl pyrophosphate (IPP), sesquiterpenes have three, diterpenes four, sesterterpenes five, triterpenes six and tetraterpenes eight IPP molecules, respectively. Polyterpenes are all terpenes containing more than eight isoprene units, which include all natural rubbers.

In Citrus spp., terpene molecules belonging to different classes are produced in leaves, fruit epidermis (flavedo) and fruit juice. These terpenes have special economic interest, as they are the main components of Citrus essential oils and some of them (carotenoids) give the Citrus juice its color. Additionally, carotenoids are well-known to be important to human health. There are several reports on the composition of terpenes in several Citrus species, mainly regarding the essential oil composition (Ruberto & Rapisarda (2002) J. Food Sci. 67:2778-2780; Sawamura, et al. (2005) J. Essen. Oil Res. 17:2-6; Verzera, et al. (2005) J. Agric. Food Chem. 53:4890-4894). The aromatic components of citrus are classified in two categories: those present in the oil from the flavedo and juice, and those soluble in the water and components of the juice. The monoterpene D-limonene is the main component of oil from the flavedo, with concentrations over 85% of the oil fraction. In addition to D-limonene, other terpenes found in the flavedo oil fraction are linalool, geraniol, citronellol, α-terpineol, valencene, myrcene, and α-pinene.

### SUMMARY OF THE INVENTION

This invention provides a method for producing a citrus terpenoid by recombinantly expressing, in a host cell that produces isopentenyl pyrophosphate (IPP) and dimethylallyl pyrophosphate (DMAPP), one or more enzymes that convert the IPP and DMAPP to a citrus terpenoid; and culturing the host cell to produce the citrus terpenoid. In some embodiments, the IPP and DMAPP are produced by a mevalonate pathway, non-mevalonate pathway, or combination thereof. In other embodiments, the one or more enzymes include terpene synthases, cytochrome P450s or a combination thereof. A recombinant host cell that produces IPP and DMAPP, and expresses one or more enzymes that convert the IPP and DMAPP to a citrus terpenoid is also provided.

### BRIEF DESCRIPTION OF THE DRAWINGS

FIG. 1 shows the steps in the mevalonate (MVA) pathway and 1-deoxy-D-xylulose-5-phosphate (DXP) pathway for generating the five carbon atoms in the basic terpenoid as well as downstream synthesis of monoterpenes and sesquiterpenes.

### DETAILED DESCRIPTION OF THE INVENTION

This invention provides, inter alia, compositions and methods for producing a citrus terpenoid in a recombinant host cell. In particular, the invention provides a method for producing a citrus terpenoid by recombinantly expressing, in a host cell that produces IPP and DMAPP, one or more enzymes that convert IPP or DMAPP to a citrus terpenoid, and culturing the host cell to produce the citrus terpenoid. Accordingly, host cells in culture include (i) one or more heterologous nucleic acids encoding one or more enzymes (e.g., terpene synthases and/or cytochrome P450 polypeptides) that convert IPP or DMAPP to a citrus terpenoid or a combination of citrus terpenoids and (ii) nucleic acid encoding polypeptides of the MVA pathway and/or DXP pathway.

For the purposes of this invention, a citrus terpenoid is intended to refer to a terpenoid produced by a plant of the genus Citrus. Citrus plants include, for example, Citrus natsudaidai (amanatsu), Citrus medica (citron), Citrus bergamia (Bergamot orange), Citrus x aurantium (bitter orange), Citrus x sinensis (blood orange), Citrus medica var. sarcodactylis (Buddha's hand), Citrus reticulata x maxima (Cam sanh), Citrus subg. Papeda, Citrus reticulate (clementine), Citrus glauca (desert lime), Citrus australasica (finger lime), Citrus paradisi (grapefruit), Citrus sphaerocarpa (kabosu), Citrus hystrix (Kaffir lime), Citrus aurantiifolia (lime), Citrus nobilis x Citrus deliciosa (kinnow), Citrus unshiu x Citrus sinensis (kiyomi), Citrus japonica (kumquat), Citrus limon (lemon), Citrus x meyeri (meyer lemon), Citrus x sinensis (orange), Citrus x latifolia (Persian lime), Citrus maxima or Citrus grandis (pumelo), Citrus limon x medica (ponderosa lemon), Citrus unshiu (mandarin), Citrus sudachi (sudachi), Citrus limetta (sweet lemon), Citrus x depressa (Taiwan tangerine), Citrus tangerine (tangerine), C. reticulata x C. sinensis (Tangor), Citrus reticulata x Citrus paradisi (Ugli fruit), and Citrus ichangensis x C. reticulate (Yuzu).

Citrus Terpenoids.

Citrus terpenoids, also referred to herein as citrus isoprenoids or citrus terpenes, are organic chemicals derived from the five-carbon isoprene unit. Citrus terpenoids of particular interest in accordance with this invention include monoterpenoids, sesquiterpenoids, diterpenoids, triterpenoids and tetraterpenoids. The recombinant host cell can be engineered to produce a single terpenoid or a mixture of two or more terpenoids. Examples of terpenoids that have been identified from Citrus are listed in Table 1.

TABLE 1
Citrus Terpenoids

| | |
|---|---|
| α-Pinene | β-Pinene |
| Camphene | Myrcene |
| Δ³-Carene | α-Phellandrene |
| β-Phellandrene | α-Terpinene |
| γ-Terpinene | D-limonene |
| Sabinene | Terpinolene |
| Linalool | Geraniol |
| Citronellol | p-Menth-1-en-9-al |
| (+)-Cis-methyl dihydrojasmonate | α-Sinensal |
| β-Sinensal | Trans,trans-farnesal |
| β-Elemol | Intermedeol |
| α-Elemene | α-Humulene |
| Cadina-1,4-diene | δ-Cadinene |
| α-Copaene | Trans-beta-farnesene |
| Selina-4,11-diene | γ-Selinene |
| γ-Muurolene | Rosefuran |
| β-Ylangene | 6-Dodecenal |
| 3-Butyl-2-thiophenecarboxyaldehyde | Ethyl 3-hydroxyhexanoate |
| 4-Butyl-2-thiophenecarboxyaldehyde | Limonen-10-yl acetate |
| p-Menth-1-en-9-yl acetate | Thymol methyl ether |
| Ethyl-3-oxo hexanoate | Rose aldehyde |
| (2e,4e)-deca-2,4-dien-1-yl acetate | 7-Methoxycoumarin |

6-Isopropenyl-4,8a-dimethyl-4a,5,6,7,8,8a-hexahydro-2(1H)-naphthalenone

In particular embodiments of this invention, the citrus terpenoid produced by the host cell is one or more of (+)-cis-methyl dihydrojasmonate (methyl 2-(3-oxo-2-pentylcyclopentyl)acetate; CAS 24851-98-7); α-sinensal ((2E,6E,9E)-2,6,10-trimethyldodeca-2,6,9,11-tetraenal; CAS 17909-77-2); β-sinensal ((2E,6E)-2,6-dimethyl-10-methylidenedodeca-2,6,11-trienal; CAS 60066-88-8); trans,trans-farnesal ((2E,6E)-3,7,11-trimethyldodeca-2,6,10-trienal; CAS 502-67-0); p-menth-1-en-9-al (2-(4-methylcyclohex-3-en-1-yl)propanal; CAS 29548-14-9); β-elemol (2-[(1R,3R,4S)-4-ethenyl-4-methyl-3-prop-1-en-2-ylcyclohexyl]propan-2-ol; CAS 32142-08-8); intermedeol ((1S,4aS,7R,8aS)-1,4a-dimethyl-7-prop-1-en-2-yl-2,3,4,5,6,7,8,8a-octahydronaphthalen-1-ol; CAS 6168-59-8); 6-isopropenyl-4,8a-dimethyl-4a,5,6,7,8,8a-hexahydro-2(1h)-naphthalenone ([4aS-(4α,6α,8α)]-4a,5,6,7,8,8a-Hexahydro-4,8a-dimethyl-6-(1-methylethenyl)-2(1H)-naphthalenone; CAS 86917-80-8); α-humulene ((1Z,4Z,8Z)-2,6,6,9-tetramethylcycloundeca-1,4,8-triene; CAS 6753-98-6); α-elemene ((6S)-6-ethenyl-6-methyl-1-propan-2-yl-3-propan-2-ylidenecyclohexene; CAS 5951-67-7); cadina-1,4-diene ((1S)-1,6-dimethyl-4-propan-2-yl-1,2,3,4,4a,7-hexahydronaphthalene; CAS 29837-12-5); δ-cadinene ((1S,8aR)-4,7-dimethyl-1-propan-2-yl-1,2,3,5,6,8a-hexahydronaphthalene; CAS 483-76-1); α-copaene (CAS 3856-25-5); selina-4,11-diene ((2S,4aS)-4a,8-dimethyl-2-prop-1-en-2-yl-2,3,4,5,6,7-hexahydro-1H-naphthalene; CAS 28290-20-2); trans-β-farnesene ((6E)-7,11-dimethyl-3-methylenedodeca-1,6,10-triene; CAS 18794-84-8); γ-selinene (8a-methyl-4-methylidene-6-propan-2-ylidene-2,3,4a,5,7,8-hexahydro-1H-naphthalene; CAS 515-17-3); γ-muurolene ((1R,4aR,8aS)-7-methyl-4-methylidene-1-propan-2-yl-2,3,4a,5,6,8a-hexahydro-1H-naphthalene; CAS 30021-74-0); rosefuran (3-methyl-2-(3-methylbut-2-enyl)furan; CAS 15186-51-3); β-ylangene ((1S,6S,7S,8S)-1-methyl-3-methylene-8-(propan-2-yl)-tricyclo[4.4.0.0(2,7)]decane; CAS 20479-06-5); 6-dodecenal (dodec-6-enal; CAS 76261-02-4); 3-butyl-2-thiophenecarboxyaldehyde (CAS 163460-99-9); 4-butyl-2-thiophenecarboxyaldehyde (CAS 163461-01-6); p-menth-1-en-9-yl acetate; (2-(4-methylcyclohex-3-en-1-yl)propyl acetate; CAS 28839-13-6); ethyl 3-hydroxyhexanoate (CAS 2305-25-1); limonen-10-yl acetate (2-(4-methylcyclohex-3-en-1-yl)prop-2-enyl acetate; CAS 15111-97-4); ethyl-3-oxo hexanoate (CAS 3249-68-1); thymol methyl ether (2-methoxy-4-methyl-1-propan-2-ylbenzene; CAS 1076-56-8); rose aldehyde (2-(4-methylcyclohex-3-en-1-yl)propanal; CAS 29548-14-9); 7-methoxycoumarin (7-methoxychromen-2-one; Herniarin; Ayapanin; CAS 531-59-9); and (2e,4e)-deca-2,4-dien-1-yl acetate (CAS 118026-67-8). In accordance with this embodiment, the amount of one or more the above-referenced citrus terpenoids accounts for 10%, 15%, 20%, 25%, 30%, 35%, 40%, 45%, 50%, 55%, 60%, 65%, 70%, 75%, 80%, 85%, 90%, 95% or 100% of the total amount (w/w) of terpenoids produced by the recombinant host cell.

In other embodiments, the recombinant host cell does not produce or produces only a minor amount of one or more of valencene, myrcene and linalool. As used herein, a minor amount of a terpenoid is an amount that does not exceed 10%, 9%, 8%, 7%, 6%, 5%, 4%, 3%, 2%, or 1% of the total amount (w/w) of terpenoids produced by the recombinant host cell.

As discussed herein, citrus terpenoid production is achieved in a host cell that includes (i) one or more heterologous nucleic acids encoding one or more enzymes that convert IPP or DMAPP to a citrus terpenoid or a combination of citrus terpenoids and (ii) nucleic acids encoding polypeptides of the MVA pathway and/or DXP pathway. By “heterologous” nucleic acid or polypeptide is meant a nucleic acid or polypeptide whose sequence is not identical to that of another nucleic acid or polypeptide naturally expressed in the same host cell. In particular, a heterologous nucleic acid or polypeptide is not identical to a wild-type nucleic acid or polypeptide that is found in the same host cell in nature.

DXP Pathway.

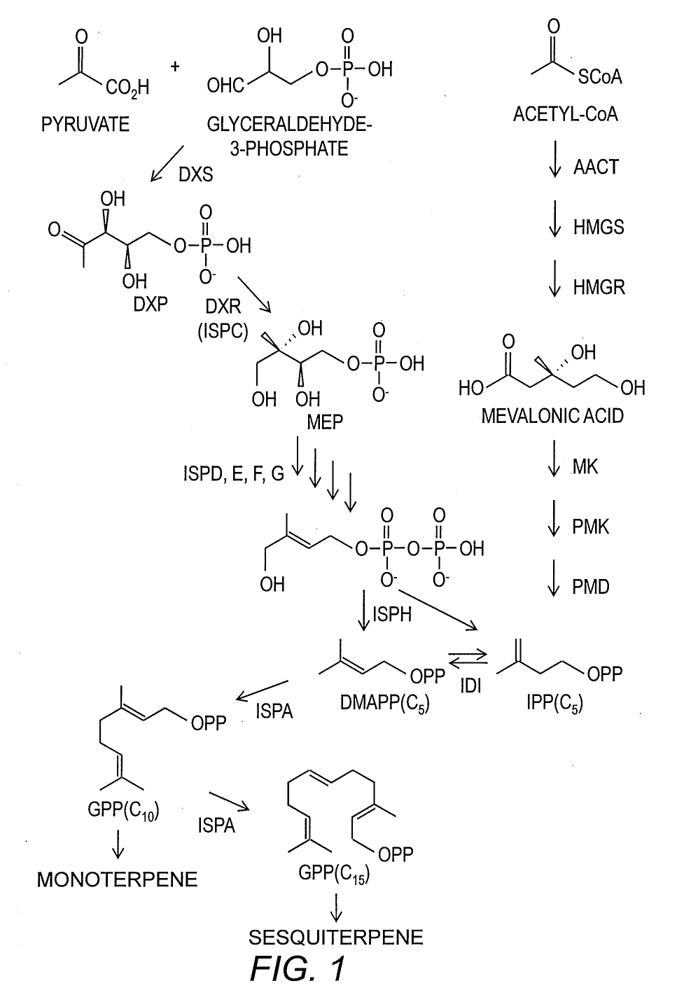

In the DXP pathway (aka, “non-mevalonate pathway,” “methylerythritol phosphate (MEP) pathway,” or “Rohmer pathway”), the five carbon atoms in the basic terpenoid unit are derived from pyruvate and glyceraldehydes-3-phosphate. See FIG. 1. Enzymes of the DXP pathway include 1-Deoxyxylulose-5-phosphate synthase (DXS; EC 2.2.1.7); 1-Deoxy-D-xylulose 5-phosphate reductoisomerase (DXR, IspC; EC 2.2.1.7); 4-Diphosphocytidyl-2C-methyl-D-erythritol synthase (MCT, IspD; EC 2.7.7.60); 4-Diphosphocytidyl-2-C-methyl-D-erythritol kinase (CMK, IspE; EC 2.7.1.148); 2C-Methyl-D-erythritol 2,4-cyclodiphosphate synthase (MCS, IspF; EC 4.6.1.12); 1-Hydroxy-2-methyl-2-(E)-butenyl 4-diphosphate synthase (HDS, IspG; EC 1.17.4.3); and 1-Hydroxy-2-methyl-2-(E)-butenyl 4-diphosphate reductase (HDR, IspH; EC 1.17.1.2).

DXS polypeptides convert pyruvate and D-glyceraldehyde-3-phosphate into 1-deoxy-D-xylulose-5-phosphate (DXP). Suitable DXS polypeptides that can be expressed in the host cell of this invention are known in the art or can be identified using conventional assays. See, e.g., Sprenger, et al. (1997) Proc. Natl. Acad. Sci. USA 94:12857-62. Exemplary DXS polypeptides and/or nucleic acids include, e.g., those found under GENBANK Accession Nos. DQ768815 (Yersinia pestis), AF143812 (Lycopersicon esculentum), Y18874 (Synechococcus PCC6301), AF035440 (E. coli), AF282878 (Pseudomonas aeruginosa), NM_121176 (Arabidopsis thaliana) and AB026631 (Streptomyces sp. CL190).

DXR polypeptides convert 1-deoxy-D-xylulose 5-phosphate (DXP) into 2-C-methyl-D-erythritol 4-phosphate (MEP). Suitable DXR polypeptides that can be expressed in the host cell of this invention are known in the art or can be identified using conventional assays. See, e.g., Hoeffler, et al. (2002) Eur. J. Biochem. 269:4446-4457. Exemplary DXR polypeptides and/or nucleic acids include, e.g., those found under GENBANK Accession Nos. AF282879 (Pseudomonas aeruginosa), AY081453 (Arabidopsis thaliana) and AJ297566 (Zea mays).

MCT polypeptides convert 2-C-methyl-D-erythritol 4-phosphate (MEP) into 4-(cytidine 5′-diphospho)-2-C-methyl-D-erythritol (CDP-Me). Suitable MCT polypeptides that can be expressed in the host cell of this invention are known in the art or can be identified using conventional assays. See, e.g., Rohdich, et al. (2000) Proc. Natl. Acad. Sci. USA 97:6451-6456. Exemplary MCT polypeptides and/or nucleic acids include, e.g., those found under GENBANK Accession Nos. AF230737 (Arabidopsis thaliana), CP000034.1 (region: 2725605 . . . 2724895; Shigella dysenteriae) and CP000036.1 (region: 2780789 . . . 2781448; Shigella boydii).

CMK polypeptides convert 4-(cytidine 5′-diphospho)-2-C-methyl-D-erythritol (CDP-ME) into 2-phospho-4-(cytidine 5′-diphospho)-2-C-methyl-D-erythritol (CDP-MEP). Suitable CMK polypeptides that can be expressed in the host cell of this invention are known in the art or can be identified using conventional assays. See, e.g., Lüttgen, et al. (2000) Proc. Natl. Acad. Sci. USA 97:1062-1067. Exemplary CMK polypeptides and/or nucleic acids include, e.g., those found under GENBANK Accession Nos. CP000036.1 (region: 1839782 . . . 1840633; Shigella boydii); AF288615 (Arabidopsis thaliana) and CP000266.1 (region: 1272480 . . . 1271629; Shigella flexneri).

MCS polypeptides convert 2-phospho-4-(cytidine 5′-diphospho)-2-C-methyl-D-erythritol (CDP-MEP) into 2-C-methyl-D-erythritol 2,4-cyclodiphoshphate (ME-CPP or cMEPP). Suitable MCS polypeptides that can be expressed in the host cell of this invention are known in the art or can be identified using conventional assays. See, e.g., Rohdich, et al. (1999) Proc. Natl. Acad. Sci. USA 96:11758-11763. Exemplary MCS polypeptides and/or nucleic acids include, e.g., those found under GENBANK Accession Nos. AE017220.1 (region: 3025667 . . . 3025216; Salmonella enterica), NM_105070 (Arabidopsis thaliana) and AE014073.1 (region: 2838621 . . . 283841; Shigella flexneri).

HDS polypeptides convert 2-C-methyl-D-erythritol 2,4-cyclodiphoshphate (ME-CPP or cMEPP) into (E)-4-hydroxy-3-methylbut-2-en-1-yl-diphosphate (HMBPP or HDMAPP). Suitable HDS polypeptides that can be expressed in the host cell of this invention are known in the art or can be identified using conventional assays. See, e.g., Kepeck, et al. (2005) J. Org. Chem. 70:9168-9174. Exemplary HDS polypeptides and/or nucleic acids include, e.g., those found under GENBANK Accession Nos. CP000034.1 (region: 2505082 . . . 2503964; Shigella dysenteriae), NM_180902 (Arabidopsis thaliana), AE008814.1 (region: 15609 . . . 14491; Salmonella typhimurium), AE014613.1 (region: 383225 . . . 384343; Salmonella enterica), AE017220.1 (region: 2678054 . . . 2676936; Salmonella enteric) and BX95085.1 (region: 3604460 . . . 3603539; Erwinia carotova).

HDR polypeptides convert (E)-4-hydroxy-3-methylbut-2-en-1-yl-diphosphate (HMBPP) into IPP and DMAPP. Suitable HDR polypeptides that can be expressed in the host cell of this invention are known in the art or can be identified using conventional assays. See, e.g., Grawert, et al. (2004) J. Am. Chem. Soc. 126:12847-12855. Exemplary HDR polypeptides and/or nucleic acids include, e.g., those found under GENBANK Accession Nos. J05090 (Saccharomyces cerevisiae) and NM_121649 (Arabidopsis thaliana), as well as those disclosed in U.S. Pat. No. 6,645,747; WO 02/095011 and WO 02/083720.

In some embodiments of the invention, the host cells express one or more DXP pathway polypeptides. In other embodiments of the invention, the host cells express 2, 3, 4, 5 or 6 DXP pathway polypeptides. While not intending to be bound by any particular theory, it is believed that increasing the amount or expression of one or more of DXS, DXR, MCT, CMK, MCS, HDS and HDR increases the flow of carbon through the DXP pathway, leading to greater terpenoid production. For example, one or more DXP pathway nucleic acids (i.e., encoding DXS, DXR, MCT, CMK, MCS, HDS, and HDR) can be introduced into the host cells to increase expression of the same. The DXS, DXR, MCT, CMK, MCS, HDS, or HDR nucleic acid may be a heterologous nucleic acid or a duplicate copy of an endogenous nucleic acid. In some embodiments, the amount of one or more of DXS, DXR, MCT, CMK, MCS, HDS, or HDR polypeptide is increased by replacing one or more endogenous DXS, DXR, MCT, CMK, MCS, HDS, or HDR promoters or regulatory regions thereof with other promoters and/or regulatory regions that result in greater transcription of one or more of DXS, DXR, MCT, CMK, MCS, HDS, or HDR nucleic acids.

In some embodiments, citrus terpenoid production can be further increased by increasing the carbon flux through the DXP pathway. In some embodiments, the carbon flux can be increased by avoiding any feedback inhibition of DXS activity by metabolites downstream of the DXP pathway and/or intermediates of other pathways that use a DXP pathway polypeptide as a substrate (e.g., DXR). In some embodiments, the feedback inhibition by some DXP pathway polypeptides (e.g., DXR) can be alleviated by rebalancing pathway enzymes and maintaining levels of HMBPP and DMAPP at concentrations below 1 to 2 mM DMAPP and 1 to 2 mM HMBPP. In some embodiments, the level of HMBPP and DMAPP are maintained below 1 mM for the duration of the fermentation run. In other embodiments, the level of HMBPP and DMAPP are maintained below 1 mM during the exponential phase of the fermentation. In other embodiments, late DXP pathway enzymes, particularly IspG and IspH, are maintained at levels consistent with minimizing the phosphorylation level of DXR.

In some embodiments, the carbon flux can be increased by expressing a DXP pathway polypeptide from a different organism that is not subject to inhibition by downstream products of the DXP pathway. In other embodiments, the carbon flux can be increased by deregulating glucose uptake. In further embodiments, the carbon flux can be increased by maximizing the balance between the precursors required for the DXP pathway. In some embodiments, the balance of the DXP pathway precursors, pyruvate and glyceraldehydes-3-phosphate (G-3-P) can be achieved by redirecting the carbon flux with the effect of elevating or lowering pyruvate or G-3-P separately. In some embodiments, the carbon flux can be increased by using a strain (containing one or more DXP pathway genes or one or more or both DXP pathway and MVA pathway genes) containing a pyruvate dehydrogenase E1 subunit variant. In some embodiments, the pyruvate dehydrogenase (PDH) E1 subunit variant has an E636Q point mutation. In some embodiments, the carbon flux can be increased by using a CRP-deleted mutant. As used herein, CRP (cAMP Receptor Protein) is a positive regulator protein activated by cyclic AMP and is required for RNA polymerase to initiate transcription of certain (catabolite-sensitive) operons of E. coli.

MVA Pathway.

In the MVA pathway (aka, “mevalonate pathway”), the five carbon atoms in the basic terpenoid unit are derived from two acetyl CoA molecules. FIG. 1. Enzymes of the MVA pathway include acetoacetyl CoA thiolase (AACT; EC. 2.3.1.9); Hydroxymethylglutaryl-CoA synthase (HMGS; EC 2.3.3.10); 3-Hydroxy-3-methylglutaryl-CoA reductase (HMGR; EC 1.1.1.34); Mevalonate kinase (MK; EC 2.7.1.36); Phosphomevalonate kinase (PMK; EC 2.7.4.2); Diphosphomevalonate decarboxylase (PMD; EC 4.1.1.33); Isopentenyl-diphosphate delta-isomerase (IDI; EC 5.3.3.2).

AACT polypeptides catalyze the condensation of two acetyl CoA molecules to yield acetoacetyl CoA. Suitable AACT polypeptides that can be expressed in the host cell of this invention are known in the art or can be identified using conventional assays. See, e.g., Hedl, et al. (2002) J. Bacteriol. 184:2116-2122. Exemplary AACT polypeptides include, e.g., those found under GENBANK Accession Nos. NC_000913 (Region: 2324131 . . . 2325315; E. coli), D49362 (Paracoccus denitrificans) and L20428 (Saccharomyces cerevisiae).

HMGS catalyzes the addition of another molecule of acetyl CoA to acetoacetyl CoA to yield HMG-CoA. Suitable HMGS polypeptides that can be expressed in the host cell of this invention are known in the art or can be identified using conventional assays. See, e.g., Sutherlin, et al. (2002) J. Bacteriol. 184:4065-4070. Exemplary HMGS polypeptides and/or nucleic acids include, e.g., those found under GENBANK Accession Nos. NC_001145 (complement: 19061 . . . 20536; Saccharomyces cerevisiae), X96617 (Saccharomyces cerevisiae), X83882 (Arabidopsis thaliana), AB037907 (Kitasatospora griseola) and BT007302 (Homo sapiens)

HMGR catalyzes the reduction of HMG-CoA to mevalonate. Suitable HMGR polypeptides that can be expressed in the host cell of this invention are known in the art or can be identified using conventional assays. See, e.g., Hedl, et al. (2002) J. Bacteriol. 184:2116-2122. Exemplary HMGR polypeptides and/or nucleic acids include, e.g., those found under GENBANK Accession Nos. NM_206548 (Drosophila melanogaster), NM_204485 (Gallus gallus), AB015627 (Streptomyces sp. KO-3988), AF542543 (Nicotiana attenuata), AB037907 (Kitasatospora griseola), AX128213 (providing the sequence encoding a truncated HMGR; Saccharomyces cerevisiae) and NC_001145 (complement: 115734 . . . 118898; Saccharomyces cerevisiae). In some embodiments, the HMGR coding region encodes a truncated form of HMGR (“tHMGR”) that lacks the transmembrane domain of wild-type HMGR. The transmembrane domain of HMGR contains the regulatory portions of the enzyme and has no catalytic activity.

MK phosphorylates mevalonate to yield phosphomevalonate. Suitable MK polypeptides that can be expressed in the host cell of this invention are known in the art or can be identified using conventional assays. See, e.g., Oulmouden & Karst (1991) Curr. Genet. 19:9-14. Exemplary MK polypeptides and/or nucleic acids include, e.g., those found under GENBANK Accession Nos. L77688 (Arabidopsis thaliana) and X55875 (Saccharomyces cerevisiae).

PMK phosphorylates phosphomevalonate to form mevalonate diphosphate. Suitable PMK polypeptides that can be expressed in the host cell of this invention are known in the art or can be identified using conventional assays. See, e.g., Tsay & Robinson (1991) Mol. Cell Biol. 11:620-631. Exemplary PMK polypeptides and/or nucleic acids include, e.g., those found under GENBANK Accession Nos. AF429385 (Hevea brasiliensis), NM_006556 (Homo sapiens) and NC_001145 (complement: 712315 . . . 713670; Saccharomyces cerevisiae).

PMD catalyzes the conversion of mevalonate diphosphate to IPP with the concomitant release of CO₂. Suitable PMD polypeptides that can be expressed in the host cell of this invention are known in the art or can be identified using conventional assays. See, e.g., Dhe-Paganon, et al. (1994) Biochemistry 33:13355-13362. Exemplary PMD polypeptides and/or nucleic acids include, e.g., those found under GENBANK Accession Nos. X97557 (Saccharomyces cerevisiae), AF290095 (Enterococcus faecium) and U49260 (Homo sapiens).

IDI catalyzes the conversion of the IPP to DMAPP. Suitable PMD polypeptides that can be expressed in the host cell of this invention are known in the art or can be identified using conventional assays. See, e.g., Anderson, et al. (1989) J. Biol. Chem. 264:19169-19175. Exemplary IDI polypeptides and/or nucleic acids include, e.g., those found under GENBANK Accession Nos. NC_000913 (region: 3031087 . . . 3031635; E. coli) and AF082326 (Haematococcus pluvialis).

In some embodiments of the invention, the host cells express one or more MVA pathway polypeptides. In other embodiments of the invention, the host cells express 2, 3, 4, 5 or 6 MVA pathway polypeptides. While not intending to be bound by any particular theory, it is believed that increasing the amount or expression of one or more of AACT, HMGS, HMGR, MK, PMK, PMD and IDI increases the flow of carbon through the MVA pathway, leading to greater terpenoid production. For example, one or more MVA pathway nucleic acids (i.e., AACT, HMGS, HMGR, MK, PMK, PMD and IDI) can be introduced into the host cells. The AACT, HMGS, HMGR, MK, PMK, PMD or IDI nucleic acid may be a heterologous nucleic acid or a duplicate copy of an endogenous nucleic acid. In some embodiments, the amount of one or more of AACT, HMGS, HMGR, MK, PMK, PMD or IDI polypeptide is increased by replacing one or more endogenous AACT, HMGS, HMGR, MK, PMK, PMD or IDI promoters or regulatory regions thereof with other promoters and/or regulatory regions that result in greater transcription of one or more of AACT, HMGS, HMGR, MK, PMK, PMD or IDI nucleic acids.

Additional MVA pathway polypeptides and/or DXP pathway polypeptides, which can be used and methods of making microorganisms (e.g., facultative anaerobes such as E. coli) encoding MVA pathway polypeptides and/or DXP pathway polypeptides are also described in WO 2009/076676; WO 2014/100726; US 2010/0184178; U.S. Pat. Nos. 8,420,360; 8,361,762; 8,470,581; 8,476,049; 8,569,026; 8,999,682 and 7,129,392, which are incorporated herein by reference.

Prenyl Transferases.

A recombinant host cell of the invention can be further modified to include a nucleic acid encoding a prenyl transferase. Prenyl transferases constitute a broad group of enzymes catalyzing the consecutive condensation of IPP resulting in the formation of prenyl diphosphates of various chain lengths. Suitable prenyl transferases include enzymes that catalyze the condensation of IPP with allylic primer substrates to form isoprenoid compounds with from about 2 isoprene units to about 6000 isoprene units or more, e.g., 2 isoprene units (Geranyl Pyrophosphate synthase), 3 isoprene units (Farnesyl pyrophosphate synthase), 4 isoprene units (geranylgeranyl pyrophosphate synthase), 5 isoprene units, isoprene units (hexadecylpyrophosphate synthase), 7 isoprene units, 8 isoprene units (phytoene synthase, octaprenyl pyrophosphate synthase), 9 isoprene units (nonaprenyl pyrophosphate synthase, 10 isoprene units (decaprenyl pyrophosphate synthase), or up to about 6000 isoprene units or more.

Suitable prenyl transferases include, but are not limited to, an E-isoprenyl diphosphate synthase, including, but not limited to, geranyl diphosphate (GPP) synthase, farnesyl diphosphate (FPP) synthase, geranylgeranyl diphosphate (GGPP) synthase, hexaprenyl diphosphate (HexPP) synthase, heptaprenyl diphosphate (HepPP) synthase, octaprenyl (OPP) diphosphate synthase, solanesyl diphosphate (SPP) synthase, decaprenyl diphosphate (DPP) synthase, chicle synthase, and gutta-percha synthase; and a Z-isoprenyl diphosphate synthase, including, but not limited to, nonaprenyl diphosphate (NPP) synthase, undecaprenyl diphosphate (UPP) synthase, dehydrodolichyl diphosphate synthase, eicosaprenyl diphosphate synthase, natural rubber synthase, and other Z-isoprenyl diphosphate synthases.

The nucleotide sequences of numerous prenyl transferases from a variety of species are known, and can be used or modified for use in generating a recombinant host cell of this invention. See, e.g., Homo sapiens farnesyl pyrophosphate synthetase mRNA (GENBANK Accession No. J05262); Saccharomyces cerevisiae farnesyl diphosphate synthetase (FPP) gene (GENBANK Accession No. J05091); Saccharomyces cerevisiae isopentenyl diphosphate:dimethylallyl diphosphate isomerase gene (GENBANK Accession No. J05090); Arabidopsis thaliana farnesyl pyrophosphate synthetase 2 (FPS2)/FPP synthetase 2/farnesyl diphosphate synthase 2 (At4g17190) mRNA (GENBANK Accession No. NM_202836); Ginkgo biloba geranylgeranyl diphosphate synthase (ggpps) mRNA (GENBANK Accession No. AY371321); Arabidopsis thaliana geranylgeranyl pyrophosphate synthase (GGPS1)/GGPP synthetase/farnesyltranstransferase (At4g36810) mRNA (GENBANK Accession No. NM_119845); Synechococcus elongatus gene for farnesyl, geranylgeranyl, geranylfarnesyl, hexaprenyl, heptaprenyl diphosphate synthase (SelF-HepPS) (GENBANK Accession No. AB016095) and the like.

In certain embodiments, the recombinant host cell of this invention includes a recombinant FPP synthase with an enhanced Kₘ value (for example, an avian FPP synthase) for DMAPP. Such high Kₘ FPP synthases have been described, for example, in Fernandez, et al. (2000) Biochemistry 39(50):15316-21. In other embodiments, the recombinant host cell of the invention can include an FPP synthase with a different temperature optimum (e.g., the thermophilic FPP synthase described in Koyama, et al. (1993) J. Biochem. 113(3):355-363), a psychrophilic FPP synthase (e.g., the FPP synthase described in Nichols, et al. (2004) J. Bad. 186:8508-8515), or an FPP synthase from a marine prokaryote (e.g., the FPP synthase described in Ranzer, et al. (2009) Mar. Biotechnol. 11:62-73). In other embodiments, the FPP synthase is a Citrus FPP synthase. Exemplary Citrus FPP synthases include, e.g., Citrus clementina FPP synthases available under Accession Nos. Ciclev10015290m, Ciclev10015517m and Ciclev10015706m from the Citrus Genome Database.

In some embodiments, an endogenous host cell gene encoding prenyl transferase is replaced by any of the alternative genes encoding a prenyl transferase described herein. In certain embodiments, a recombinant prenyl transferase gene is placed under the control of an inducible or a constitutive promoter. In other embodiments, a recombinant prenyl transferase gene is expressed on a multicopy plasmid. In still another embodiment, a recombinant prenyl transferase gene is integrated into a chromosome of the host cells.

Citrus Terpenoid Synthesis.

Citrus terpenoid synthesis is achieved using terpene synthase and/or cytochrome P450 polypeptides. The particular enzymes expressed by the host cell will be dependent upon the citrus terpenoid or terpenoids to be produced by the host cell. In one embodiment, the citrus terpenoid(s) of interest is produced using one or more terpene synthases. In another embodiment, the citrus terpenoid(s) of interest is produced using one or more cytochrome P450 polypeptides. In other embodiments, the citrus terpenoid(s) of interest is produced using one or more terpene synthases in combination with one or more cytochrome P450 polypeptides.

Terpene Synthases.

As used herein, the term “terpene synthase” refers to any enzyme that enzymatically modifies monoprenyl diphosphates such as IPP and DMAPP, or polyprenyl pyrophosphates (i.e., compounds containing two or more prenyl groups) to produce terpenoid compounds. The term “terpene synthase” includes enzymes that catalyze the conversion of a prenyl diphosphate into an isoprenoid. X-ray structural analyses indicate that terpene synthases generally adopt two kinds of folds, ionization-initiated and protonation-initiated terpene synthases. Ionization-initiated terpene synthases have been designated type I terpene synthases. The best known structural motif of the type I terpene synthase family is an aspartate-rich region, D-D-X-X-(D/E) (SEQ ID NO:1), found in virtually all isolated plant terpene synthases as well as in isoprenyl diphosphate synthases and microbial terpene synthases. Site-directed mutagenesis as well as X-ray structural analysis reveal that this region is involved in binding divalent metal ions, which in turn interact with the diphosphate moiety of the substrate (Starks, et al. (1997) Science 277:1815-1820; Lesburg, et al. (1997) Science 277:1820-1824; Tarshis, et al. (1994) Biochemistry 33:10871-10877; Tarshis, et al. (1996) Proc. Natl. Acad. Sci. USA 93:15018-15023; Cane, et al. (1996) Biochemistry 35:12369-12376; Cane, et al. (1996) J. Am. Chem. Soc. 118:8499-8500). The location of the D-D-X-X-(D/E) (SEQ ID NO:1) motif at the entrance of the catalytic site appears to be critical in positioning the substrate for catalysis. Mutations in this region frequently lead to decreased catalytic activity and the appearance of abnormal products that can be attributed to altered substrate binding (Cane, et al. (1996) Biochemistry 35:12369-12376; Cane, et al. (1996) J. Am. Chem. Soc. 118:8499-8500; Rynkiewicz, et al. (2002) Biochemistry 41:1732-1741; Seemann, et al. (2002) J. Am. Chem. Soc. 124:7681-7689; Prosser, et al. (2004) Arch. Biochem. Biophys. 432:136-144). However, a naturally occurring variant of the D-D-X-X-(D/E) motif, a N-D-X-X-D (SEQ ID NO:2) sequence in the fully active (+) germacrene-synthase from goldenrod, also exhibits catalytic activity.

An additional metal cofactor binding motif located on the opposite site of the active site entry has also been described (Christianson (2006) Chem. Rev. 106:3412-3442). This motif, designated NSE/DTE motif, has apparently evolved from a second aspartate-rich motif conserved in prenyl transferases to form a consensus sequence of (L/V)-(V/L/A)-(N/D)-D-(L/I/V)-X-(S/T)-X-X-X-E (SEQ ID NO:3; Cane & Kang (2000) Arch. Biochem. Biophys. 376:354-364; Christianson (2006) Chem. Rev. 106:3412-3442). Both the D-D-X-X-(D/E) (SEQ ID NO:1) motif and the NSE/DTE motif bind a trinuclear magnesium cluster involved in fixation of the pyrophosphate substrate. Whereas the D-D-X-X-(D/E) (SEQ ID NO:1) motif is highly conserved throughout almost all plant terpene synthases, the NSE/DTE motif appears to be less well conserved. In some sesquiterpene synthases, the NSE/DTE motif is replaced by a second D-D-X-X-(D/E) (SEQ ID NO:1) motif (Steele, et al. (1998) J. Biol. Chem. 273: 2078-2089) which was also shown to be involved in catalysis (Little & Croteau (2002) Arch. Biochem. Biophys. 402:120-135).

About 35 amino acids upstream of the D-D-X-X-(D/E) (SEQ ID NO:1) motif in type I enzymes is a highly conserved R-R-(X)₈-W (SEQ ID NO:4) motif that is implicated in the complexation of the diphosphate function after ionization of the substrate preventing nucleophilic attack on any of the carbocationic intermediates (Starks, et al. (1997) Science 277:1815-1820). The R-R-(X)₈₋W (SEQ ID NO:4) motif has been found to be absolutely conserved in most Citrus sequences that resemble typical monoterpene synthases (Dornelas & Mazzafera (2007) Genet. Mol. Biol. 30:832-840). Deletion studies on the limonene synthase of Mentha spicata indicate that all amino acids N-terminal to this point were dispensable for enzyme activity (Williams, et al. (1998) Biochemistry 37:12213-12220). However, deletion of the tandem arginine motif renders the limonene synthase unable to accept geranyl diphosphate as a substrate. Since the enzyme is still able to convert linalyl diphosphate to limonene, this suggests that the tandem arginine motif might participate in the isomerization of GPP to a cyclizable intermediate, such as the linalyl cation (Williams, et al. (1998) Biochemistry 37:12213-12220). In keeping with this suggestion, the tandem arginine motif can be absent in monoterpene synthases producing only acyclic compounds, which do not require isomerization.

Compared to type I, type II terpene synthases are protonation-initiated. The corresponding active sites reside between β/γ domains, both of which exhibit an α-barrel fold in which a D-X-D-D (SEQ ID NO:5) motif in the β domain provides the proton donor that triggers initial carbocation formation (Christianson (2006) Chem. Rev. 106:3412-3442). The γ fold exhibits a similar topology with p fold.

Accordingly, in some embodiments, a host cell of this invention recombinantly expresses (i) one or more heterologous nucleic acids encoding one or more terpene synthase having an amino acid sequence that includes the sequence D-D-X-X-(D/E) (SEQ ID NO:1), N-D-X-X-D (SEQ ID NO:2), (L/V)-(V/L/A)-(N/D)-D-(L/I/V)-X-(S/T)-X-X-X-E (SEQ ID NO:3), R-R-(X)₈₋W (SEQ ID NO:4), D-X-D-D (SEQ ID NO:5), or a combination thereof, which convert IPP or DMAPP to a citrus terpenoid or a combination of citrus terpenoids, and (ii) nucleic acids and polypeptides of the MVA pathway and/or DXP pathway.

The terpene synthase and/or nucleic acids and polypeptides of the MVA pathway and/or DXP pathway of this invention can be obtained from a plant (e.g., an angiosperm or gymnosperm), alga, fungus or bacterium. By way of illustration, the terpene synthase is obtained from a non-Citrus sp. including, but not limited to, Vitis vinifera (grape), Pogostemon cablin (patchouli), Santalum album (white sandalwood), Gossypium hirsutum (upland cotton), G. arboretum (tree cotton), Artemesia annua (sweet wormwood), Ixeridium dentatum, Solidago canadensis (goldenrod), Solanum lycopersicum (tomato), S. habrochaites (wild tomato), Nicotiana tabacum (tobacco), Ocimum basilicum (sweet basil), Fabiana imbricate, Cucumis sativus (Cucumber), Cucumis melo (Muskmelon), Centella asiatica, Populus trichocarpa x deltoids, Actinidia deliciosa (Kiwi), Medicago truncatula (Barrel medic), Zea mays (Maize), Oryza sativa (Rice), Zea mays huehuetenanangensis, Zingiber zerumbet (Shampoo ginger), Zingiber officinale (Ginger), Elaeis oleifera (Oil palm), Magnolia grandiflora (Southern magnolia), Picea abies (Norway spruce), Picea sitchensis (Sitka spruce), Pinus sylvestris (Scots pine), Abies grandis (Grand fir), Pinus taeda (Loblolly pine), Lavandula angustifolia (Lavender), Antirrhinum majus (Garden snapdragon), Malus domestica (Apple), Solanum tuberosum (Potato), Hyoscyamus muticus (Egyptian henbane), Nicotiana attenuate, Capsicum annuum (Red pepper), Perilla frutescens (Beefsteak plant), Marrubium vulgare (White horehound), Mentha piperita (Peppermint), Arabidopsis thaliana (Mouse-ear cress), Ricinus communis (Castor bean), Lactuca sativa (Garden lettuce), Crepidiastrum sonchifolium, Cichorium intybus (Chicory) and Helianthus annuus (Common sunflower).

By way of illustration, terpene synthase enzymes from non-Citrus species include, for example, those listed in Table 3 and sesquiterpene synthases such as that from Pogostemon cablin (UniProt Accession No. Q49SP4).

TABLE 3

| | | |
|---|---|---|
| | Accession | |
| Organism | No.¹ | Product |

α-Copaene synthases (EC 4.2.3.133)

| | | |
|---|---|---|
| Phyla dulcis | J7LP58 | α-copaene, δ-cadinene |
| Ricinus communis | B9S9Z3 | α-copaene, δ-cadinene |

α-Pinene synthases (EC 4.2.3.119)

| | | |
|---|---|---|
| Abies grandis | O24475 | (−)-α-pinene, (−)-β- |
| | | pinene |
| | Q9M7C9 | (−)-α-pinene, |
| | | terpinolene, (−)- |
| | | limonene, (−)-β-pinene |
| | | plus minor products |
| | Q948Z0 | (−)-α-pinene, (−)- |
| | | camphene |
| Artemisia annua | Q94G53 | (−)-α-pinene, (−)-β- |
| | | pinene |
| Gossypium hirsutum | U5N0S4 | α-pinene, β-pinene, β- |
| | | phellandrene |
| Picea sitchensis | Q6XDB5 | (−)-α-pinene, (−)-β- |
| | | pinene |
| Pinus banksiana | R9QMW5 or | (−)-α-pinene, (−)-β- |
| | R9QMY9 | pinene |
| Pinus contorta | R9QMR3 | (−)-α-pinene, (−)-β- |
| | | pinene |
| Pinus taeda | Q84KL6 | (−)-α-pinene, (−)-β- |
| | | pinene, camphene, |
| | | limonene |
| Pseudotsuga | Q4QSN3 | (−)-α-pinene, (−)- |
| menziessii | | camphene, plus minor |
| | | products |

β-Pinene synthases (EC 4.2.3.120)

| | | |
|---|---|---|
| Abies grandis | O24475 | (−)-α-pinene, (−)-β- |
| | | pinene |
| Artemisia annua | Q94G53 | (−)-α-pinene, (−)-β- |
| | | pinene |
| Picea sitchensis | Q6XDB5 | (−)-α-pinene, (−)-β- |
| | | pinene |
| Pinus banksiana | R9QMW5, | (−)-α-pinene, (−)-β- |
| | R9QMY9 | pinene |

Camphene synthases (EC 4.2.3.117)

| | | |
|---|---|---|
| Abies grandis | Q948Z0 | (−)-α-pinene, (−)- |
| | | camphene |
| Pseudotsuga | Q4QSN3 | (−)-α-pinene, (−)- |
| menziessii | | camphene, plus minor |
| | | products |

Myrcene synthases (EC 4.2.3.15)

| | | |
|---|---|---|
| Abies grandis | O24474 | myrcene |
| Arabidopsis thaliana | Q9ZUH4 | β-myrcene, (E)-β-ocimene, |
| | | and minor amounts of (+)- |
| | | limonene, (−)-limonene, |
| | | 2-carene |
| Ips pini | Q58GE8 | myrcene |
| Humulus lupulus | EU760349 | myrcene |

Δ³-Carene synthases (EC 4.2.3.107)

| | | |
|---|---|---|
| Picea sitchensis | F1CKI9 | (+)-car-3-ene, |
| | | (−)-sabinene, terpinolene |
| Picea abies | Q84SM8 | (+)-car-3-ene, |
| | | terpinolene |
| Salvia stenophylla | Q8L5J7 | (+)-3-carene, (−)- |
| | | limonene, myrcene, 4- |
| | | carene, beta-phellandrene |

α-Phellandrene synthases

| | | |
|---|---|---|
| Vitis vinifera | E5GAG2 | α-phellandrene |
| Solanum pennellii | G5CV35 | α-phellandrene |

β-Phellandrene synthases (EC 4.2.3.52)

| | | |
|---|---|---|
| Abies grandis | Q9M7D1 | β-phellandrene |
| Solanum lycopersicum | C1K5M3 | β-phellandrene |

α-Terpinene synthases (EC 4.2.3.115)
γ-Terpinene synthases (EC 4.2.3.114)

| | | |
|---|---|---|
| Origanum vulgare | E2E2P0 | γ-terpinene and minor |
| | | products |
| Origanum syriacum | G3LTY3 | γ-terpinene |
| Thymus caespititius | R4JHV6 | γ-terpinene, plus minor |
| | | products α-terpinene and |
| | | α-thujene |
| Salvia officinalis | O81193 | γ-terpinene, sabinene, |
| | | terpinolene, limonene, |
| | | myrcene |

D-limonene synthases (EC 4.2.3.20)

| | | |
|---|---|---|
| Agastache rugosa | Q940E7 | (R)-limonene |
| Lavandula | Q2XSC6 | (R)-limonene, |
| angustifolia | | terpinolene, (1R,5S)- |
| | | camphene, (1R,5R)-(+)-α- |
| | | pinene, betamyrcene |
| Schizonepeta | Q9FUW5 | (R)-limonene |
| tenuifolia | | |

Sabinene synthases (EC 4.2.3.109)

| | | |
|---|---|---|
| Arabidopsis thaliana | P0DI76 | 1,8-cineole, (−)- |
| | | sabinene, myrcene and |
| | | minor amounts of α- |
| | | thujene, α-pinene, (2)- |
| | | β-pinene, myrcene, |
| | | limonene, β-ocimene, |
| | | terpinolene |
| Salvia officinalis | O81193 | γ-terpinene, sabinene, |
| | | terpinolene, limonene, |
| | | myrcene |
| Picea sitchensis | F1CKJ1 | sabinene |

Terpinolene synthases (EC 4.2.3.113)

| | | |
|---|---|---|
| Abies grandis | Q9M7D0 | terpinolene, α-pinene, |
| | | limonene, β-pinene |
| Lavandula | Q2XSC6 | (R)-(+)-limonene, |
| angustifolia | | terpinolene, camphene, α- |
| | | pinene, betamyrcene |
| Picea abies | Q84SM8 | (+)-car-3-ene, |
| | | terpinolene |
| Pseudotsuga menziesii | Q4QSN6 | terpinolene, plus minor |
| | | products |
| Salvia officinalis | O81193 | γ-terpinene, sabinene, |
| | | terpinolene, limonene, |
| | | myrcene |
| Picea sitchensis | F1CKI9 | (+)-car-3-ene, |
| | | (−)-sabinene, terpinolene |

Linalool synthases (EC 4.2.3.25/26)

| | | |
|---|---|---|
| Actinidia arguta | D4N3A0 | linalool |
| Actinidia polygama | D4N3A1 | linalool |
| Perilla setoyensis | C0KWV3 | linalool |
| Mentha × piperita | Q8H2B4 | linalool |
| subsp. citrate | | |
| Solanum lycopersicum | Q1XBU5 | linalool |

Geraniol synthases (EC 3.1.7.11)

| | | |
|---|---|---|
| Cinnamomum tenuipile | Q8GUE4 | geraniol |
| Perilla citriodora | Q4JHG3 | geraniol |
| Catharanthus roseus | J9PZR5 | geraniol |
| Ocimum basilicum | Q6USK1 | geraniol |
| Perilla frutescens | Q308N0 | geraniol |
| Perilla setoyensis | C0KWV4 | geraniol |

β-Elemol synthases

| | | |
|---|---|---|
| Santalum spicatum | E3W208 | β-elemol, guaiol, |
| | | bulnesol |

α-Humulene synthases (EC 4.2.3.104)

| | | |
|---|---|---|
| Gossypium hirsutum | K7PRF2 | p-caryophyllene and α- |
| | | humulene |
| | U5N1F1 | β-caryophyllene and α- |
| | | humulene |
| Helianthus annuus | Q4U3F6 | α-humulene |
| Solanum habrochaites | G8H5M8 | α-humulene and β- |
| | | caryophyllene |
| Solanum lycopersicum | D5KXD2 | α-humulene |
| Zingiber zerumbet | B1B1U3 | α-humulene |

δ-Cadinene synthases (EC 4.2.3.13)

| | | |
|---|---|---|
| Gossypium arboretum | Q39761 | δ-cadinene |
| Gossypium hirsutum | P93665 | δ-cadinene |
| Cucumis melo | B2KSJ5 | δ-cadinene |
| Helianthus annuus | Q4U3F6 | δ-cadinene |
| Thapsia garganica | K4L9M2 | δ-cadinene |
| Phyla dulcis | J7LP58 | α-copaene, δ-cadinene |
| Ricinus communis | B9S9Z3 | α-copaene, δ-cadinene |

Trans-beta-farnesene (EC 4.2.3.47)

| | | |
|---|---|---|
| Artemisia annua | Q9FXY7 | (E)-β-farnesene |
| Zea mays | Q84ZW8 | (E)-β-farnesene |
| Streptomyces | Q9K498 | (E)-β-farnesene, (3E,6E)- |
| coelicolor | | α-farnesene, (3Z,6E)-α- |
| | | farnesene, neroliol, and |
| | | farnesol |
| Zea diploperennis | C7E5V9 | (E)-β-farnesene |
| Zea perennis | C7E5W0 | (E)-β-farnesene |
| Mentha piperita | O48935 | (E)-β-farnesene |
| Oryza sativa | Q0J7R9 | 7-epi-sesquithujene, (E)- |
| | | α-bergamotene, |
| | | sesquiabinene A, (E)-β- |
| | | farnesene, gamma- |
| | | curcumene, zingiberene, |
| | | β-bisabolene, β- |
| | | sesquiphellandrene, (E)- |
| | | γ-bisabolene |
| Pseudotsuga menziesii | AAX07265 | (E)-β-farnesene |

Selina-4,11-diene synthases

| | | |
|---|---|---|
| Vitis vinifera | HM807406 | selina-411-diene, |
| | | intermedeol |

γ-Muurolene Synthases (EC 4.2.3.126)

| | | |
|---|---|---|
| Artemisia annua | Q9FXY7 | γ-muurolene |
| Coprinopsis cinerea | A8NE23 | β-elemene, γ-muurolene, |
| | | germacrene D, and δ- |
| | | cadinene |
| Santalum album | B5A435 | γ-muurolene |

¹UniProt/GENBANK

Ideally, the terpene synthase is obtained from Citrus. Citrus terpene synthases include, e.g., (E)-β-Farnesene synthase obtained from Citrus ichangensis x C. reticulate (UniProt Accession No. Q94JS8) as well as synthases available from the Citrus sinensis Annotation Project (Wang, et al. (2014) PLOS ONE 9(1):e87723; Ding, et al. (2014) BMC Plant Biol. 14(1):213) under ID Nos. Cs1g10750, Cs2g08460, Cs2g08510, Cs2g08520, Cs2g08540, Cs2g08550, Cs2g08560, Cs2g08650, Cs2g22100, Cs2g22150, Cs2g23470, Cs2g24110, Cs2g24130, Cs2g24530, Cs4g04630, Cs4g04660, Cs4g04680, Cs4g04730, Cs4g04740, Cs4g08260, Cs4g11320, Cs4g12050, Cs4g12080, Cs4g12090, Cs4g12110, Cs4g12120, Cs4g12350, Cs4g12400, Cs4g12450, Cs4g12490, Cs5g12880, Cs5g12900, Cs5g15530, Cs5g23510, Cs5g23540, Cs5g31210, Cs5g33990, Cs5g34010, Cs6g13250, Cs7g16690, Cs8g05710, Cs8g20920, Cs8g20950, Cs9g13320, Cs9g16490, Cs9g16510, orange1.1t00017, orange1.1t03278, orange1.1t05393, orange1.1t05421, and orange1.1t05525, orange1.1t05697. Additional terpene synthases are provided in the CitEST database (Dornelas & Mazzafera (2007) Genet. Mol. Biol. 30:832-840) and include, e.g., Citrus aurantium terpene synthases available under accession numbers. CA26C1002055E0 and CA26C1002043B01; Citrus aurantifolia terpene synthases available under accession numbers CG32C1003043D03 and CG32C1003065H02; Citrus reticulata terpene synthases available under accession numbers CR05C1100013H05, CR05C370013B09, CR05C1100017G04, CR05C3700046D07, CR05C3702070D10, CR05C3700064C07, CR05C3700084E04, CR05C3700094A06, CR0503702081D05, CR05C1102019H10, CR05C1103053D10, CR0503700058F06, CR05C3700006A05 and CR05C3700094H08; Citrus sinensis terpene synthases available under accession numbers CS00C3700044G09, CS0001100110A04, CS00C3700005G05, CS00C1100123A05, CS00C3701012D12, CS00C370022006, CS0003702030D10, CS0003701079G08, CS0003700039G08, CS00C3702001D11, CS00C3701092G09, CS00C1101038D02, CS00C3700070C12, CS00C3700069C03, CS00C3701092G08, CS00C3700108H08 an d CS00C1100019F09; Citrus latifolia terpene synthases available under accession numbers LT33C1003037F04, LT33C1003049B05, LT33C1003103G03, LT33C1003065F01, LT33C1003003E07, LT33C1003029A07, LT33C1003043E09 and LT33C1003056E04; and Citrus trifoliate terpene synthases available under accession numbers PT11C2300054G01, PT11C1901024B09, PT11C1900035F10, PT11C2300012H06 and PT11C1901080G07.

Cytochrome P450 Polypeptides.

By “P450 polypeptide,” “cytochrome P450,” or “P450” is meant a polypeptide that contains a heme-binding domain and shows a CO absorption spectra peak at 450 nm according to standard methods. See, e.g., Omura & Sato (1964) J. Biol. Chem. 239:2370-2378. Such P450s may include, without limitation, hydroxylation activity, oxidation activity, epoxidation activity, dehydration activity, dehydrogenation activity, dehalogenation activity, isomerization activity, alcohol oxidation activity, aldehyde oxidation activity dealkylation activity, and C—C bond cleavage activity. These reactions have been described by, e.g., Sono, et al. ((1996) Chem. Rev. 96:2841-2887; see, e.g., FIG. 3). In certain embodiments, the cytochrome P450 polypeptide has a heme-binding domain containing the amino acid sequence G-R-R-X-C-P-(A/G)(SEQ ID NO:6)

Exemplary cytochrome P450 polypeptides include, but are not limited to, members of the CYP71 family (e.g., CYP71D20, CYP71D21, CYP71D-A4, CYP71D55 and CYP71AV1, or modified versions thereof), members of the CYP73 family (e.g., CYP73A27 and CYP73A28, or modified versions thereof) and members of the CYP92 family (e.g., CYP92A5, or a modified version thereof). See U.S. Pat. Nos. 8,445,231 and 8,759,632, incorporated herein by reference, as well as WO 2015/030681. Other examples of suitable P450 polypeptides that can be modified to exhibit the desired activity include, but are not limited to, limonene-6-hydroxylase (see, e.g., GENBANK Accession Nos. AY281025 and AF124815); 5-epi-aristolochene dihydroxylase (see, e.g., GENBANK Accession No. AF368376); δ-cadinene-8-hydroxylase (see, e.g., GENBANK Accession No. AF332974); taxadiene-5a-hydroxylase (see, e.g., GENBANK Accession Nos. AY289209, AY959320, and AY364469); and ent-kaurene oxidase (see, e.g., GENBANK Accession No. AF047719).

The accession numbers of exemplary enzymes and their corresponding sequences are available from public databases such as GENBANK, UnitProt, CitEST database and the Citrus sinensis Annotation Project. The KEGG database also contains the amino acid and nucleic acid sequences of numerous exemplary enzyme and nucleic acid sequences, particularly with respect to the terpene synthase, cytochrome P450, prenyl transferase, MVA pathway and/or DXP pathway polypeptides and nucleic acids.

Nucleic acids encoding terpene synthases, cytochrome P450s, prenyl transferases, enzymes of the DXP pathway and/or enzymes of the MVA pathway can be isolated using standard methods. Methods of obtaining desired nucleic acids from a source organism of interest (such as a bacterial genome) are common and well-known in the art of molecular biology (see, for example, WO 2004/033646 and references cited therein, particularly with respect to the isolation of nucleic acids of interest). For example, if the sequence of the nucleic acid is known (such as any of the known nucleic acids described herein), suitable genomic libraries may be created by restriction endonuclease digestion and may be screened with probes complementary to the desired nucleic acid sequence. Once the sequence is isolated, the DNA may be amplified using standard primer directed amplification methods such as polymerase chain reaction (PCR) (U.S. Pat. No. 4,683,202, which is hereby incorporated by reference in its entirety, particularly with respect to PCR methods) to obtain amounts of DNA suitable for transformation using appropriate vectors. Alternatively, the terpene synthase, cytochrome P450, prenyl transferase, DXP pathway, and/or MVA pathway nucleic acids can be chemically synthesized using standard methods.

Additional nucleic acids encoding terpene synthases, cytochrome P450s, prenyl transferases, enzymes of the DXP pathway and/or enzymes of the MVA pathway, which may be suitable for use in the compositions and methods described herein can be identified using standard methods. For example, cosmid libraries of the chromosomal DNA of organisms (e.g., Citrus sp.) known to produce a citrus terpenoid naturally can be constructed in organisms such as E. coli, and then screened for terpenoid production. In particular, cosmid libraries may be created where large segments of genomic DNA (35-45 kb) are packaged into vectors and used to transform appropriate hosts. Cosmid vectors are unique in being able to accommodate large quantities of DNA. Generally cosmid vectors have at least one copy of the cos DNA sequence which is needed for packaging and subsequent circularization of the heterologous DNA. In addition to the cos sequence, these vectors also contain an origin of replication such as ColEI and drug resistance markers such as a nucleic acid resistant to ampicillin or neomycin. Methods of using cosmid vectors for the transformation of suitable bacterial hosts are well described in Sambrook et al. ((1989) Molecular Cloning: A Laboratory Manual, 2^{nd} ed., Cold Spring Harbor), particularly with respect to transformation methods.

Additional methods for obtaining a terpene synthase, cytochrome P450, prenyl transferase, DXP pathway, and/or MVA pathway nucleic acids include screening a metagenomic library by assay (such as the headspace assay (see, for example, U.S. Pat. No. 8,288,148, which is hereby incorporated by reference in its entirety) or by PCR using primers directed against nucleotides encoding for a length of conserved amino acids (for example, at least 3 conserved amino acids). Conserved amino acids can be identified by aligning amino acid sequences of a known terpene synthase, cytochrome P450, prenyl transferase, DXP pathway, or MVA pathway nucleic acid. Conserved amino acids can be identified based on aligned sequences of known polypeptides. An organism found to produce a citrus terpenoid naturally can be subjected to standard protein purification methods (which are well known in the art) and the resulting purified polypeptide can be sequenced using standard methods. Other methods are found in the literature (see, for example, Julsing, et al. (2007) Appl. Microbiol. Biotechnol. 75:1377-84; Withers, et al. (2007) Appl. Microbiol. Biotechnol. 73(19):6277-83).

In some embodiments, the host cell may be a cell that naturally produces IPP or DMAPP. In one embodiment, the host cell naturally produces IPP or DMAPP using the DXP pathway. In an alternative embodiment, the host cell naturally produces IPP or DMAPP using the MVA pathway. In some embodiments, the host cell has been modified for enhanced production of IPP or DMAPP. In other embodiments, the host cell may be a cell that does not naturally produce IPP or DMAPP. In accordance with this embodiment, the host cell is modified to heterologously express one or more enzymes of the DXP and/or MVA pathway. In further embodiments, one or more of the terpene synthase or cytochrome P450 polypeptides or nucleic acids used in the synthesis of a citrus terpenoid are heterologous to the host cell.

To facilitate expression, nucleic acids used to generate a recombinant host cell can be modified such that the nucleotide sequences reflect the codon preference for the particular host cell. For example, the nucleotide sequence will, in some embodiments, be modified for yeast codon preference. See, e.g., Bennetzen & Hall (1982) J. Biol. Chem. 257(6):3026-3031. As another non-limiting example, the nucleotide sequence will be modified for E. coli codon preference. See, e.g., Gouy & Gautier (1982) Nucleic Acids Res. 10(22):7055-7074; Eyre-Walker (1996) Mol. Biol. Evol. 13(6):864-872. See also Nakamura, et al. (2000) Nucleic Acids Res. 28(1):292.

The coding sequence of any known MVA or DXP pathway enzyme may be altered in various ways known in the art to generate targeted changes in the amino acid sequence of the encoded enzyme. The amino acid sequence of a variant MVA or DXP pathway enzyme will in some embodiments be substantially similar to the amino acid sequence of any known MVA or DXP pathway enzyme, i.e., will differ by at least one amino acid, and may differ by at least two, at least 5, at least 10, or at least 20 amino acids, but typically not more than about fifty amino acids. The sequence changes may be substitutions, insertions or deletions. For example, as described herein, the nucleotide sequence can be altered for the codon bias of a particular host cell. In addition, one or more nucleotide sequence differences can be introduced that result in conservative amino acid changes in the encoded protein.

In certain embodiments, a nucleic acid used to generate a recombinant host cell encodes a MVA or DXP pathway enzyme that has at least about 45%, at least about 50%, at least about 55%, at least about 57%, at least about 60%, at least about 65%, at least about 70%, at least about 75%, at least about 80%, at least about 85%, at least about 90%, at least about 95%, at least about 98%, or at least about 99% amino acid sequence identity to a known or naturally-occurring MVA or DXP pathway enzyme.

Constructs.

The present invention further provides recombinant vectors or constructs encoding one or more nucleic acid molecules described herein. In some embodiments, a recombinant vector provides for amplification of a nucleic acid. In other embodiments, a recombinant vector provides for production (i.e., expression) of an encoded terpene synthase, cytochrome P450, prenyl transferase, MVA pathway enzyme or DXP pathway enzyme in a eukaryotic cell, in a prokaryotic cell, or in a cell-free transcription/translation system. Suitable expression vectors include, but are not limited to, baculovirus vectors, bacteriophage vectors, plasmids, phagemids, cosmids, fosmids, bacterial artificial chromosomes, viral vectors (e.g., viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli, yeast, and plant cells).

Any vector capable of accepting a nucleic acid is contemplated as a suitable recombinant vector for the purposes of the invention. The vector may be any circular or linear length of DNA that either integrates into the host genome or is maintained in episomal form. Vectors may require additional manipulation or particular conditions to be efficiently incorporated into a host cell (e.g., many expression plasmids), or can be part of a self-integrating, cell specific system (e.g., a recombinant virus). The vector is in some embodiments functional in a prokaryotic cell, where such vectors function to propagate the recombinant vector and/or provide for expression of a nucleic acid. The vector is in some embodiments functional in a eukaryotic cell, where the vector will in many embodiments be an expression vector.

Numerous suitable expression vectors are known to those of skill in the art, and many are commercially available. The following vectors are provided by way of example for bacterial host cells: PBLUESCRIPT (Stratagene, San Diego, Calif.); pQE vectors (Qiagen); pNH vectors; lambda-ZAP vectors (Stratagene); pTrc (Amann, et al. (1988) Gene 69:301-315); pTrc99a, pKK223-3, pDR540, and pRIT2T (Pharmacia). The following vectors are provided by way of example for eukaryotic host cells: pXT1, pSG5 (Stratagene), pSVK3, pBPV, pMSG, and pSVLSV40 (Pharmacia). However, any other plasmid or other vector may be used so long as it is compatible with the host cell.

A recombinant vector will, in many embodiments, contain one or more selectable marker genes to provide a phenotypic trait for selection of transformed host cells. Suitable selectable markers include, but are not limited to, dihydrofolate reductase, neomycin resistance for eukaryotic cell culture; and tetracycline or ampicillin resistance in prokaryotic host cells such as E. coli.

In general, nucleic acids to be expressed are operably linked to one or more regulatory elements including transcriptional and/or translational control elements such as promoters, enhancers, terminators, and cis-elements. In some embodiments, expression of one or more nucleic acids is controlled by an inducible promoter. In other embodiments, expression of one or more nucleic acids is controlled by a constitutive promoter.

Suitable promoters for use in prokaryotic host cells include, but are not limited to, a bacteriophage T7 RNA polymerase promoter; a trp promoter; a lac operon promoter; a trc promoter; a tac promoter; a hybrid promoter, e.g., lac/tac hybrid promoter, a tac/trc hybrid promoter, a trp/lac promoter, or a T7/lac promoter; a lacZ promoter, an P_{BAD} promoter; in vivo regulated promoters, such as an ssaG promoter or a related promoter (see, e.g., US 2004/0131637); a pagC promoter (Pulkkinen & Miller (1991) J. Bacteriol. 173(1):86-93); a nirB promoter (Harborne, et al. (1992) Mol. Micro. 6:2805-2813); a sigma70 promoter, e.g., a consensus sigma70 promoter (see, e.g., GENBANK Accession Nos. AX798980, AX798961, and AX798183); a stationary phase promoter, e.g., a dps promoter or a spy promoter; a promoter derived from the pathogenicity island SPI-2 (see, e.g., WO 96/17951); an actA promoter (see, e.g., Shetron-Rama, et al. (2002) Infect. Immun. 70:1087-1096); an rpsM promoter (see, e.g., Valdivia & Falkow (1996) Mol. Microbiol. 22:367-378); a tet promoter; an SP6 promoter (see, e.g., Melton, et al. (1984) Nucl. Acids Res. 12:7035-7056); a low-phosphate repressible promoter (see, e.g., US 2016/0017342) and the like.

Non-limiting examples of suitable eukaryotic promoters include CMV immediate early, HSV thymidine kinase, early and late SV40, LTRs from retrovirus, and mouse metallothionein-I. In some embodiments, e.g., for expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHO5 promoter, a CUP1 promoter, a GAL7 promoter, a MET25 promoter, a MET3 promoter, a glucose isomerase promoter (see, e.g., U.S. Pat. No. 7,132,527), and the like. Selection of the appropriate vector and promoter is well within the level of ordinary skill in the art. The expression vector may also contain a ribosome binding site for translation initiation and a transcription terminator. The expression vector may also include appropriate sequences for amplifying expression.

In certain embodiments, nucleic acids encoding a terpene synthase or cytochrome P450 are operably linked to an inducible promoter. Inducible promoters are well-known in the art. Suitable inducible promoters include, but are not limited to, the pL of bacteriophage λ; Plac; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (see, e.g., Guzman, et al. (1995) J. Bacteria 177:4121-4130); a xylose-inducible promoter, e.g., Pxyl (see, e.g., Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; a heat-inducible promoter, e.g., heat inducible lambda P_{L} promoter, a promoter controlled by a heat-sensitive repressor (e.g., CI857-repressed lambda-based expression vectors; see, e.g., Hoffmann, et al. (1999) FEMS Microbiol Lett. 177(2):327-34); and the like.

In yeast, a number of vectors containing constitutive or inducible promoters may be used. For a review see, Current Protocols in Molecular Biology (1988) Vol. 2, Ed. Ausubel, et al., Greene Publish. Assoc. & Wiley Interscience, Ch. 13; Grant, et al. (1987) Expression and Secretion Vectors for Yeast, in Methods in Enzymology, Eds. Wu & Grossman, Acad. Press, NY, Vol. 153, pp. 516-544; Glover (1986) DNA Cloning, Vol. II, IRL Press, Wash., D.C., Ch. 3; Bitter (1987) Heterologous Gene Expression in Yeast, Methods in Enzymology, Eds. Berger & Kimmel, Acad. Press, NY, Vol. 152, pp. 673-684; and The Molecular Biology of the Yeast Saccharomyces (1982) Eds. Strathern et al., Cold. Spring Harbor Press, Vols. I and II. A constitutive yeast promoter such as ADH or LEU2 or an inducible promoter such as GAL may be used. Alternatively, vectors may be used which promote integration of foreign DNA sequences into the yeast chromosome.

In some embodiments, a nucleic acid or vector includes a promoter or other regulatory element(s) for expression in a plant cell. Non-limiting examples of suitable constitutive promoters that are functional in a plant cell is the cauliflower mosaic virus 35S promoter, a tandem 35S promoter (Kay, et al. (1987) Science 236:1299), a cauliflower mosaic virus 19S promoter, a nopaline synthase gene promoter (Singer, et al. (1990) Plant Mol. Biol. 14:433; An (1986) Plant Physiol. 81:86), an octopine synthase gene promoter, and a ubiquitin promoter. Suitable inducible promoters that are functional in a plant cell include, but are not limited to, a phenylalanine ammonia-lyase gene promoter, a chalcone synthase gene promoter, a pathogenesis-related protein gene promoter, a copper-inducible regulatory element (Mett, et al. (1993) Proc. Natl. Acad. Sci. USA 90:4567-4571; Furst, et al. (1988) Cell 55:705-717); tetracycline and chlor-tetracycline-inducible regulatory elements (Gatz, et al. (1992 Plant J. 2:397-404); Röder, et al. (1994) Mol. Gen. Genet. 243:32-38; Gatz (1995) Meth. Cell Biol. 50:411-424); ecdysone-inducible regulatory elements (Christopherson, et al. (1992) Proc. Natl. Acad. Sci. USA 89:6314-6318; Kreutzweiser, et al. (1994) Ecotoxicol. Environ. Safety 28:14-24); heat shock-inducible regulatory elements (Takahashi, et al. (1992) Plant Physiol. 99:383-390; Yabe, et al. (1994) Plant Cell Physiol. 35:1207-1219; Ueda, et al. (1996) Mol. Gen. Genet. 250:533-539); and lac operon elements, which are used in combination with a constitutively expressed lac repressor to confer, for example, IPTG-inducible expression (Wilde, et al. (1992) EMBO J. 11:1251-1259); a nitrate-inducible promoter derived from the spinach nitrite reductase gene (Back, et al. (1991) Plant Mol. Biol. 17:9); a light-inducible promoter, such as that associated with the small subunit of RuBP carboxylase or the LHCP gene families (Feinbaum, et al. (1991) Mol. Gen. Genet. 226:449; Lam & Chua (1990) Science 248:471); a light-responsive regulatory element as described in US 2004/0038400; a salicylic acid-inducible regulatory element (Uknes, et al. (1993) Plant Cell 5:159-169; Bi, et al. (1995) Plant J. 8:235-245); plant hormone-inducible regulatory elements (Yamaguchi-Shinozaki, et al. (1990) Plant Mol. Biol. 15:905; Kares, et al. (1990) Plant Mol. Biol. 15:225); and human hormone-inducible regulatory elements such as the human glucocorticoid response element (Schena, et al. (1991) Proc. Natl. Acad. Sci. USA 88:10421).

Plant tissue-selective regulatory elements also can be included in a nucleic acid or vector of the invention. Suitable tissue-selective regulatory elements, which can be used to ectopically express a nucleic acid in a single tissue or in a limited number of tissues, include, but are not limited to, a xylem-selective regulatory element, a tracheid-selective regulatory element, a fiber-selective regulatory element, a trichome-selective regulatory element (see, e.g., Wang et al. (2002) J. Exp. Botany 53:1891-1897), a glandular trichome-selective regulatory element, and the like.

Vectors that are suitable for use in plant cells are known in the art, and any such vector can be used to introduce a nucleic acid into a plant host cell. Suitable vectors include, e.g., a Ti plasmid of Agrobacterium tumefaciens or a Ri plasmid of A. rhizogenes. The Ti or Ri plasmid is transmitted to plant cells on infection by Agrobacterium and is stably integrated into the plant genome (Schell (1987) Science 237:1176-83). Also suitable for use is a plant artificial chromosome, as described in, e.g., U.S. Pat. No. 6,900,012.

Host Cells.

The present invention provides recombinant host cells, i.e., host cells that have been genetically modified with a nucleic acid or a recombinant vector. In many embodiments, a recombinant host cell is an in vitro host cell. In other embodiments, a recombinant host cell is an in vivo host cell. In other embodiments, a recombinant host cell is part of a multicellular organism.

Host cells are in many embodiments unicellular organisms, or are grown in culture as single cells. In some embodiments, the host cell is a eukaryotic cell. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some embodiments, the host cell is a eukaryotic cell other than a plant cell.

In other embodiments, the host cell is a plant cell. Plant cells include cells of monocotyledons (“monocots”) and dicotyledons (“dicots”). Exemplary plant cells include, but are not limited to Zea mays, Arabidopsis thaliana, Nicotiana tabacum, Brassica sp., Oryza sativa, Solanum tuberosum, and the like.

In other embodiments, the host cell is a prokaryotic cell. Suitable prokaryotic cells include, but are not limited to, any of a variety of laboratory strains of Escherichia coli, Lactobacillus sp., Salmonella sp., Shigella sp., and the like. See, e.g., Carrier, et al. (1992) J. Immunol. 148:1176-1181; U.S. Pat. No. 6,447,784; and Sizemore, et al. (1995) Science 270:299-302. Examples of Salmonella strains which can be used in the present invention include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the host cell is Escherichia coli.

To generate a recombinant host cell, nucleic acids encoding a terpene synthase and/or cytochrome P450, and optionally a prenyl transferase and/or one or more enzymes of the MVA and/or DXP pathway is introduced stably or transiently into a parent host cell, using established techniques, including, but not limited to, electroporation, calcium phosphate precipitation, DEAE-dextran mediated transfection, liposome-mediated transfection, and the like. For stable transformation, a nucleic acid will generally further include a selectable marker, e.g., any of several well-known selectable markers such as neomycin resistance, ampicillin resistance, tetracycline resistance, chloramphenicol resistance, kanamycin resistance, and the like.

In some embodiments, a recombinant host cell is a plant cell. A recombinant plant cell is useful for producing a selected citrus terpenoid compound in in vitro plant cell culture. Guidance with respect to plant tissue culture may be found in, for example: Plant Cell and Tissue Culture (1994) Vasil & Thorpe Eds., Kluwer Academic Publishers; and Plant Cell Culture Protocols, Methods in Molecular Biology (1999) Hall, Ed., Humana Press.

Recombinant Host Cells.

In accordance with this invention, a recombinant host cell harbors an expression vector, where the expression vector includes nucleic acids encoding one or more terpene synthases and/or a cytochrome P450s. In some embodiments, a recombinant host cell is a host cell that does not normally synthesize IPP, DMAPP or mevalonate via a MVA pathway. Accordingly, in some embodiments, the host cell is genetically modified with an expression vector including nucleic acids encoding one or more terpene synthases and/or cytochrome P450s; and the host cell is genetically modified with one or more heterologous nucleic acids encoding AACT, HMGS, HMGR, MK, PMK, and MPD (and optionally also IPP isomerase). In other embodiments, the host cell is genetically modified with an expression vector including nucleic acids encoding one or more terpene synthases and/or cytochrome P450s; and the host cell is genetically modified with one or more heterologous nucleic acids encoding MK, PMK, MPD (and optionally also IPP isomerase). In still other embodiments, the host cell is genetically modified with an expression vector including nucleic acids encoding one or more terpene synthases and/or cytochrome P450s; and the host cell is genetically modified with one or more heterologous nucleic acids encoding AACT, HMGS, HMGR, MK, PMK, MPD, IPP isomerase, and a prenyl transferase. In still further embodiments, the host cell is genetically modified with an expression vector including nucleic acids encoding one or more terpene synthases and/or cytochrome P450s; and the host cell is genetically modified with one or more heterologous nucleic acids encoding MK, PMK, MPD, IPP isomerase, and a prenyl transferase. In some embodiments, a recombinant host cell is one that normally synthesizes IPP or mevalonate via a MVA pathway, e.g., the host cell is one that includes an endogenous MVA pathway. In some of these embodiments, the host cell is a yeast cell, e.g., Saccharomyces cerevisiae.

In other embodiments, a recombinant host cell is a host cell that does not normally synthesize IPP or DMAPP via a DXP pathway. Accordingly, in some embodiments, the host cell is genetically modified with an expression vector including nucleic acids encoding one or more terpene synthases and/or cytochrome P450s; and the host cell is genetically modified with one or more heterologous nucleic acids encoding DXS, DXR, MCT, CMK, MCS, HDS, and HDR (and optionally also IPP isomerase). In other embodiments, the host cell is genetically modified with an expression vector including nucleic acids encoding one or more terpene synthases and/or cytochrome P450s; and the host cell is genetically modified with one or more heterologous nucleic acids encoding DXS, DXR, MCT, CMK, MCS, HDS, HDR, IPP isomerase, and a prenyl transferase.

Additional Genetic Modifications.

In some embodiments, a recombinant host cell is one that is genetically modified to include one or more nucleic acids encoding one or more terpene synthases and/or cytochrome P450s; and that is further genetically modified to achieve enhanced production of a terpene biosynthetic pathway intermediate, and/or that is further genetically modified such that an endogenous terpene biosynthetic pathway gene is functionally disabled. The term “functionally disabled,” as used herein in the context of an endogenous terpene biosynthetic pathway gene, refers to a genetic modification of a terpene biosynthetic pathway gene, which modification results in production of a gene product encoded by the gene that is produced at below normal levels, and/or is non-functional.

Genetic modifications that enhance production of an endogenous terpene biosynthetic pathway intermediate include, but are not limited to, genetic modifications that result in a reduced level and/or activity of a phosphotransacetylase in the host cell. The intracellular concentration of a terpene biosynthetic pathway intermediate is enhanced by increasing the intracellular concentration of acetyl-CoA. E. coli secretes a significant fraction of intracellular acetyl-CoA in the form of acetate into the medium. Deleting the gene encoding phosphotransacetylase, pta, the first enzyme responsible for transforming acetyl-CoA into acetate, reduces acetate secretion. Genetic modifications that reduce the level and/or activity of phosphotransacetylase in a prokaryotic host cell are particularly useful where the recombinant host cell is one that is genetically modified with a nucleic acid encoding one or more MVA pathway gene products.

In some embodiments, a genetic modification that results in a reduced level of phosphotransacetylase in a prokaryotic host cell is a genetic mutation that functionally disables the prokaryotic host cell's endogenous pta gene encoding the phosphotransacetylase. The pta gene can be functionally disabled in any of a variety of ways, including insertion of a mobile genetic element (e.g., a transposon, etc.); deletion of all or part of the gene, such that the gene product is not made, or is truncated and is non-functional in converting acetyl-CoA to acetate; mutation of the gene such that the gene product is not made, or is truncated and is non-functional in converting acetyl-CoA to acetate; deletion or mutation of one or more control elements that control expression of the pta gene such that the gene product is not made; and the like.

In some embodiments, a recombinant host cell is one that is genetically modified to include one or more nucleic acids encoding MVA pathway gene product(s); and that is further genetically modified such that an endogenous DXP biosynthetic pathway gene is functionally disabled. In other embodiments, a recombinant host cell is one that is genetically modified to include one or more nucleic acids encoding DXP pathway gene product(s); and that is further genetically modified such that an endogenous MVA pathway gene is functionally disabled.

In some embodiments, where the recombinant host cell is a prokaryotic host cell that is genetically modified with nucleic acids encoding one or more MVA pathway gene products, the host cell will be further genetically modified such that one or more endogenous DXP pathway genes is functionally disabled. DXP pathway genes that can be functionally disabled include one or more of the genes encoding any of the following DXP gene products: DXS, DXR, MCT, CMK, MCS, HDS and HDR.

An endogenous DXP pathway gene can be functionally disabled in any of a variety of ways, including insertion of a mobile genetic element (e.g., a transposon, etc.); deletion of all or part of the gene, such that the gene product is not made, or is truncated and is enzymatically inactive; mutation of the gene such that the gene product is not made, or is truncated and is enzymatically non-functional; deletion or mutation of one or more control elements that control expression of the gene such that the gene product is not made; and the like.

The present invention further provides compositions including a recombinant host cell. Such a composition includes a recombinant host cell, and will in some embodiments include one or more further components, which components are selected based in part on the intended use of the recombinant host cell. Suitable components include, but are not limited to, salts, buffers, stabilizers, protease-inhibiting agents, nuclease-inhibiting agents, cell membrane- and/or cell wall-preserving compounds, e.g., glycerol or dimethylsulfoxide, nutritional media appropriate to the cell, and the like. In some embodiments, the cells are lyophilized.

In some embodiments, a nucleic acid or an expression vector is used as a transgene to generate a transgenic plant that produces the encoded terpene synthase and/or cytochrome P450. Thus, the present invention further provides a transgenic plant, wherein the plant includes a transgene harboring a nucleic acid encoding one or more terpene synthases and/or cytochrome P450s. In some embodiments, the transgenic plant is homozygous for the genetic modification. In other embodiments, the transgenic plant is heterozygous for the genetic modification.

In some embodiments, a transgenic plant produces a transgene-encoded polypeptide that exhibits terpene synthase or cytochrome P450 activity in an amount that is at least about 50%, at least about 2-fold, at least about 5-fold, at least about 10-fold, at least about 25-fold, at least about 50-fold, or at least about 100-fold, or higher, than the amount of the polypeptide produced by a control plant, e.g., a non-transgenic plant (a plant that does not include the transgene encoding the polypeptide) of the same species.

Methods of introducing exogenous nucleic acids into plant cells are well-known in the art. Suitable methods include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e. in vitro, ex vivo, or in vivo).

Plants that can be genetically modified include grains, forage crops, fruits, vegetables, oil seed crops, palms, forestry, and vines. Specific examples of plants that can be modified include, but are not limited to, maize, banana, peanut, field peas, sunflower, tomato, canola, tobacco, wheat, barley, oats, potato, soybeans, cotton, carnations, sorghum, lupin and rice. Other examples include Artemisia annua, or other plants known to produce isoprenoid compounds of interest.

Also provided by this invention are transformed plant cells, tissues, plants and products that contain the transformed plant cells. A feature of the transformed cells, and tissues and products that include the same is the presence of a nucleic acid integrated into the genome, and production by plant cells of a polypeptide that exhibits terpene synthase or cytochrome P450 activity. Recombinant plant cells of the present invention are useful as populations of recombinant cells, or as a tissue, seed, whole plant, stem, fruit, leaf, root, flower, stem, tuber, grain, animal feed, a field of plants, and the like.

Method for Producing a Citrus Terpenoid.

The present invention also provides a method of producing a citrus terpenoid. In some embodiments, the method generally involves culturing a recombinant host cell in a suitable medium, wherein said host cell is genetically modified with a nucleic acid encoding one or more terpene synthases and/or cytochrome P450s. In other embodiments, the method generally involves maintaining a transgenic plant under conditions that favor production of the encoded terpene synthases and/or cytochrome P450s. Production of the terpene synthases and/or cytochrome P450s results in production of the citrus terpenoid. For example, in some embodiments, the method generally involves culturing a recombinant host cell in a suitable medium, wherein said host cell is genetically modified with a nucleic acid encoding a terpene synthase or cytochrome P450. Production of the terpene synthase or cytochrome P450 results in production of the citrus terpenoid. Typically, the method is carried out in vitro, although in vivo production of a citrus terpenoid is also contemplated. In some of these embodiments, the host cell is a eukaryotic cell, e.g., a yeast cell. In other embodiments, the host cell is a prokaryotic cell. In some of these embodiments, the host cell is a plant cell. In some embodiments, the method is carried out in a transgenic plant.

Any carbon source can be used to cultivate the host cells. The term “carbon source” refers to one or more carbon-containing compounds capable of being metabolized by a host cell or organism. For example, the cell medium used to cultivate the recombinant host cells may include any carbon source suitable for maintaining the viability or growing the host cells. In some embodiments, the carbon source is a carbohydrate (such as monosaccharide, disaccharide, oligosaccharide, or polysaccharides), invert sugar (e.g., enzymatically treated sucrose syrup), glycerol, glycerin (e.g., a glycerin byproduct of a biodiesel or soap-making process), dihydroxyacetone, one-carbon source, oil (e.g., a plant or vegetable oil such as corn, palm, or soybean oil), animal fat, animal oil, fatty acid (e.g., a saturated fatty acid, unsaturated fatty acid, or polyunsaturated fatty acid), lipid, phospholipid, glycerolipid, monoglyceride, diglyceride, triglyceride, polypeptide (e.g., a microbial or plant protein or peptide), renewable carbon source (e.g., a biomass carbon source such as a hydrolyzed biomass carbon source), yeast extract, component from a yeast extract, polymer, acid, alcohol, aldehyde, ketone, amino acid, succinate, lactate, acetate, ethanol, or any combination of two or more of the foregoing. In some embodiments, the carbon source is product of photosynthesis, including, but not limited to, glucose.

Exemplary monosaccharides include glucose and fructose; exemplary oligosaccharides include lactose and sucrose, and exemplary polysaccharides include starch and cellulose. Exemplary carbohydrates include C6 sugars (e.g., fructose, mannose, galactose, or glucose) and C5 sugars (e.g., xylose or arabinose). In some embodiments, the cell medium includes a carbohydrate as well as a carbon source other than a carbohydrate (e.g., glycerol, glycerine, dihydroxyacetone, one-carbon source, oil, animal fat, animal oil, fatty acid, lipid, phospholipid, glycerolipid, monoglyceride, diglyceride, triglyceride, renewable carbon source, or a component from a yeast extract). In some embodiments, the cell medium includes a carbohydrate as well as a polypeptide (e.g., a microbial or plant protein or peptide). In some embodiments, the microbial polypeptide is a polypeptide from yeast or bacteria. In some embodiments, the plant polypeptide is a polypeptide from soy, corn, canola, jatropha, palm, peanut, sunflower, coconut, mustard, rapeseed, cottonseed, palm kernel, olive, safflower, sesame, or linseed.

Typically, the concentration of the carbohydrate is at least or about 5 grams per liter of broth (g/L, wherein the volume of broth includes both the volume of the cell medium and the volume of the cells), such as at least or about 10, 15, 20, 30, 40, 50, 60, 80, 100, 150, 200, 300, 400, or more g/L. In some embodiments, the concentration of the carbohydrate is between about 50 and about 400 g/L, such as between about 100 and about 360 g/L, between about 120 and about 360 g/L, or between about 200 and about 300 g/L. In some embodiments, this concentration of carbohydrate includes the total amount of carbohydrate that is added before and/or during the culturing of the host cells.

In some embodiments, the cells are cultured under limited glucose conditions. By “limited glucose conditions” is meant that the amount of glucose that is added is less than or about 105% (such as about 100%) of the amount of glucose that is consumed by the cells. In particular embodiments, the amount of glucose that is added to the culture medium is approximately the same as the amount of glucose that is consumed by the cells during a specific period of time. In some embodiments, the rate of cell growth is controlled by limiting the amount of added glucose such that the cells grow at the rate that can be supported by the amount of glucose in the cell medium. In some embodiments, glucose does not accumulate during the time the cells are cultured. In various embodiments, the cells are cultured under limited glucose conditions for greater than or about 1, 2, 3, 5, 10, 15, 20, 25, 30, 35, 40, 50, 60, or 70 hours. In various embodiments, the cells are cultured under limited glucose conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited glucose conditions may allow more favorable regulation of the cells.

In alternative embodiments, the cells are cultured in the presence of an excess of glucose. In accordance with this embodiment, the amount of glucose that is added is greater than about 105% (such as about or greater than 110, 120, 150, 175, 200, 250, 300, 400, or 500%) or more of the amount of glucose that is consumed by the cells during a specific period of time. In some embodiments, glucose accumulates during the time the cells are cultured.

Exemplary lipids are any substance containing one or more fatty acids that are C4 and above fatty acids that are saturated, unsaturated, or branched. Exemplary oils are lipids that are liquid at room temperature. In some embodiments, the lipid contains one or more C4 or above fatty acids (e.g., contains one or more saturated, unsaturated, or branched fatty acid with four or more carbons). In some embodiments, the oil is obtained from soy, corn, canola, jatropha, palm, peanut, sunflower, coconut, mustard, rapeseed, cottonseed, palm kernel, olive, safflower, sesame, linseed, oleagineous microbial cells, Chinese tallow, or any combination of two or more of the foregoing. Exemplary fatty acids include compounds of the formula RCOOH, where “R” is a hydrocarbon. Exemplary unsaturated fatty acids include compounds where “R” includes at least one carbon-carbon double bond. Exemplary unsaturated fatty acids include, but are not limited to, oleic acid, vaccenic acid, linoleic acid, palmitelaidic acid, and arachidonic acid. Exemplary polyunsaturated fatty acids include compounds where “R” includes a plurality of carbon-carbon double bonds. Exemplary saturated fatty acids include compounds where “R” is a saturated aliphatic group. In some embodiments, the carbon source includes one or more C₁₂-C₂₂ fatty acids, such as a C₁₂ saturated fatty acid, a C₁₄ saturated fatty acid, a C₁₆ saturated fatty acid, a C₁₈ saturated fatty acid, a C₂₀ saturated fatty acid, or a C₂₂ saturated fatty acid. In an exemplary embodiment, the fatty acid is palmitic acid. In some embodiments, the carbon source is a salt of a fatty acid (e.g., an unsaturated fatty acid), a derivative of a fatty acid (e.g., an unsaturated fatty acid), or a salt of a derivative of fatty acid (e.g., an unsaturated fatty acid). Suitable salts include, but are not limited to, lithium salts, potassium salts, sodium salts, and the like. Di- and triglycerols are fatty acid esters of glycerol.

The concentration of the lipid, oil, fat, fatty acid, monoglyceride, diglyceride, or triglyceride may be at least or about 1 gram per liter of broth (g/L, wherein the volume of broth includes both the volume of the cell medium and the volume of the cells), such as at least or about 5, 10, 15, 20, 30, 40, 50, 60, 80, 100, 150, 200, 300, 400, or more g/L. In some embodiments, the concentration of the lipid, oil, fat, fatty acid, monoglyceride, diglyceride, or triglyceride is between about 10 and about 400 g/L, such as between about 25 and about 300 g/L, between about 60 and about 180 g/L, or between about 75 and about 150 g/L. In some embodiments, the concentration includes the total amount of the lipid, oil, fat, fatty acid, monoglyceride, diglyceride, or triglyceride that is added before and/or during the culturing of the host cells. In some embodiments, the carbon source includes both (i) a lipid, oil, fat, fatty acid, monoglyceride, diglyceride, or triglyceride and (ii) a carbohydrate, such as glucose. In some embodiments, the ratio of the lipid, oil, fat, fatty acid, monoglyceride, diglyceride, or triglyceride to the carbohydrate is about 1:1 on a carbon basis (i.e., one carbon in the lipid, oil, fat, fatty acid, monoglyceride, diglyceride, or triglyceride per carbohydrate carbon). In particular embodiments, the amount of the lipid, oil, fat, fatty acid, monoglyceride, diglyceride, or triglyceride is between about 60 and 180 g/L, and the amount of the carbohydrate is between about 120 and 360 g/L.

Exemplary microbial polypeptide carbon sources include one or more polypeptides from yeast or bacteria. Exemplary plant polypeptide carbon sources include one or more polypeptides from soy, corn, canola, jatropha, palm, peanut, sunflower, coconut, mustard, rapeseed, cottonseed, palm kernel, olive, safflower, sesame, or linseed.

Exemplary renewable carbon sources include cheese whey permeate, cornsteep liquor, sugar beet molasses, barley malt, and components from any of the foregoing. Exemplary renewable carbon sources also include glucose, hexose, pentose and xylose present in biomass, such as corn, switchgrass, sugar cane, cell waste of fermentation processes, and protein by-product from the milling of soy, corn, or wheat. In some embodiments, the biomass carbon source is a lignocellulosic, hemicellulosic, or cellulosic material such as, but are not limited to, a grass, wheat, wheat straw, bagasse, sugar cane bagasse, soft wood pulp, corn, corn cob or husk, corn kernel, fiber from corn kernels, corn stover, switch grass, rice hull product, or a by-product from wet or dry milling of grains (e.g., corn, sorghum, rye, triticate, barley, wheat, and/or distillers grains). Exemplary cellulosic materials include wood, paper and pulp waste, herbaceous plants, and fruit pulp. In some embodiments, the carbon source includes any plant part, such as stems, grains, roots, or tubers. In some embodiments, all or part of any of the following plants are used as a carbon source: corn, wheat, rye, sorghum, triticate, rice, millet, barley, cassava, legumes, such as beans and peas, potatoes, sweet potatoes, bananas, sugarcane, and/or tapioca. In some embodiments, the carbon source is a biomass hydrolysate, such as a biomass hydrolysate that includes both xylose and glucose or that includes both sucrose and glucose. In some embodiments, the renewable carbon source (such as biomass) is pretreated before it is added to the cell culture medium. In some embodiments, the pretreatment includes enzymatic pretreatment, chemical pretreatment, or a combination of both enzymatic and chemical pretreatment (see, for example, Farzaneh, et al. (2005) Bioresource Technology 96(18):2014-2018; U.S. Pat. Nos. 6,176,176; 6,106,888). In some embodiments, the renewable carbon source is partially or completely hydrolyzed before it is added to the cell culture medium.

In some embodiments, the concentration of the carbon source (e.g., a renewable carbon source) is equivalent to at least or about 0.1, 0.5, 1, 1.5 2, 3, 4, 5, 10, 15, 20, 30, 40, or 50% glucose (w/v). The equivalent amount of glucose can be determined by using standard HPLC methods with glucose as a reference to measure the amount of glucose generated from the carbon source. In some embodiments, the concentration of the carbon source (e.g., a renewable carbon source) is equivalent to between about 0.1 and about 20% glucose, such as between about 0.1 and about 10% glucose, between about 0.5 and about 10% glucose, between about 1 and about 10% glucose, between about 1 and about 5% glucose, or between about 1 and about 2% glucose.

In some embodiments, the carbon source includes yeast extract or one or more components of yeast extract. In some embodiments, the concentration of yeast extract is at least 1 gram of yeast extract per liter of broth (g/L, wherein the volume of broth includes both the volume of the cell medium and the volume of the cells), such at least or about 5, 10, 15, 20, 30, 40, 50, 60, 80, 100, 150, 200, 300, or more g/L. In some embodiments, the concentration of yeast extract is between about 1 and about 300 g/L, such as between about 1 and about 200 g/L, between about 5 and about 200 g/L, between about 5 and about 100 g/L, or between about 5 and about 60 g/L. In some embodiments, the concentration includes the total amount of yeast extract that is added before and/or during the culturing of the host cells. In some embodiments, the carbon source includes both yeast extract (or one or more components thereof) and another carbon source, such as glucose. In some embodiments, the ratio of yeast extract to the other carbon source is about 1:5, about 1:10, or about 1:20 (w/w).

Additionally, the carbon source may also be one-carbon substrates such as carbon dioxide, or methanol. Glycerol production from single carbon sources (e.g., methanol, formaldehyde, or formate) has been reported in methylotrophic yeasts (Yamada, et al. (1989) Agric. Biol. Chem. 53(2)541-543) and in bacteria (Hunter, et al. (1985) Biochemistry 24:4148-4155). These organisms can assimilate single carbon compounds, ranging in oxidation state from methane to formate, and produce glycerol.

In some embodiments, cells are cultured in, a standard medium containing physiological salts and nutrients (see, e.g., Pourquie, et al. (1988) Biochemistry and Genetics of Cellulose Degradation, Aubert et al., Eds., Academic Press, pp. 71-86 and Ilmen, et al. (1997) Appl. Environ. Microbiol. 63:1298-1306). Exemplary growth media are common commercially prepared media such as Luria Bertani (LB) broth, Sabouraud Dextrose (SD) broth, or Yeast medium (YM) broth. Other defined or synthetic growth media may also be used, and the appropriate medium for growth of particular host cells are known by someone skilled in the art of microbiology or fermentation science.

Depending on the host cell used, the amount of citrus terpenoid produced can be increased by adding yeast extract to the cell culture medium. For example, the amount of citrus terpenoid produced can be linearly proportional to the amount of yeast extract in the cell medium. As such, increasing the amount of yeast extract in the presence of glucose can result in more citrus terpenoid being produced than increasing the amount of glucose in the presence of yeast extract. Also, increasing the amount of yeast extract can allow the cells to produce a high level of citrus terpenoid for a longer length of time and improved the health of the cells.

Depending on the culture medium in which the host cell is cultured, and depending on whether the host cell synthesizes IPP via a DXP pathway or via a MVA pathway, the host cell will in some embodiments include further genetic modifications. For example, in some embodiments, the host cell is one that does not have an endogenous MVA pathway, e.g., the host cell is one that does not normally synthesize IPP or mevalonate via a MVA pathway. For example, in some embodiments, the host cell is one that does not normally synthesize IPP via a mevalonate pathway, and the host cell is genetically modified with one or more nucleic acids encoding two or more enzymes in the MVA pathway, an IPP isomerase, a prenyl transferase, a terpene synthase and/or cytochrome P450. Culturing such a host cell provides for production of the MVA pathway enzymes, the IPP isomerase, the prenyl transferase, the terpene synthase, and/or cytochrome P450. Production of the MVA pathway enzymes, the IPP isomerase, the prenyl transferase, the terpene synthase and/or cytochrome P450 results in production of a citrus terpenoid. In many embodiments, the prenyl transferase is an FPP synthase, which generates a sesquiterpene substrate for a sesquiterpene oxidase encoded by a nucleic acid; and production of the sesquiterpene oxidase results in oxidation of the sesquiterpene substrate in the host cell. Any nucleic acids encoding the MVA pathway enzymes, the IPP isomerase, the prenyl transferase, the terpene synthase and/or cytochrome P450 are suitable for use.

In some of the above-described embodiments, the host cell is genetically modified with one or more nucleic acids encoding two or more MVA pathway enzymes, wherein the two or more MVA pathway enzymes include MK, PMK, and MPD, and the host cell is cultured in medium that includes mevalonate. In other embodiments, the two or more MVA pathway enzymes include acetoacetyl CoA thiolase, HMGS, HMGR, MK, PMK, and MPD.

Materials and methods suitable for the maintenance and growth of cell cultures are well known in the art. Exemplary techniques may be found in Manual of Methods for General Bacteriology (1994) Gerhardt et al., Eds, American Society for Microbiology, Washington, D.C. or Brock (1989) in Biotechnology: A Textbook of Industrial Microbiology, Second Edition, Sinauer Associates, Inc., Sunderland, Mass. In some embodiments, the cells are cultured in a culture medium under conditions permitting the expression of one or more terpene synthases, cytochrome P450s, DXP pathway polypeptides, MVA pathway polypeptides, and/or prenyl transferase polypeptide encoded by nucleic acids inserted into the host cells.

Standard cell culture conditions can be used to culture the cells (see, for example, WO 2004/033646 and references cited therein). Cells are grown and maintained at an appropriate temperature, gas mixture, and pH (such as at about 20 to about 37° C., at about 6% to about 84% CO₂, and at a pH between about 5 to about 9). In some embodiments, cells are grown at 35° C. in an appropriate cell medium. In some embodiments, cultures are cultured at approximately 28° C. in appropriate medium in shake cultures or fermenters until desired amount of citrus terpenoid production is achieved. In some embodiments, the pH ranges for fermentation are between about pH 5.0 to about pH 9.0 (such as about pH 6.0 to about pH 8.0 or about 6.5 to about 7.0). Reactions may be performed under aerobic, anoxic, or anaerobic conditions based on the requirements of the host cells.

Recombinant host cells can be grown using any known mode of fermentation, such as batch, fed-batch, or continuous processes. In some embodiments, a batch method of fermentation is used. Classical batch fermentation is a closed system where the composition of the media is set at the beginning of the fermentation and is not subject to artificial alterations during the fermentation. Thus, at the beginning of the fermentation the cell medium is inoculated with the desired host cells and fermentation is permitted to occur adding nothing to the system. Typically, however, “batch” fermentation is batch with respect to the addition of carbon source and attempts are often made at controlling factors such as pH and oxygen concentration. In batch systems, the metabolite and biomass compositions of the system change constantly until the time the fermentation is stopped. Within batch cultures, cells moderate through a static lag phase to a high growth log phase and finally to a stationary phase where growth rate is diminished or halted. In some embodiments, cells in log phase are responsible for the bulk of the citrus terpenoid production. In some embodiments, cells in stationary phase produce citrus terpenoids.

A variation on the standard batch system can also be used, such as the Fed-Batch system. Fed-Batch fermentation processes include a typical batch system with the exception that the carbon source is added in increments as the fermentation progresses. Fed-Batch systems are useful when catabolite repression is apt to inhibit the metabolism of the cells and where it is desirable to have limited amounts of carbon source in the cell medium. Fed-batch fermentations may be performed with the carbon source (e.g., glucose) in a limited or excess amount. Measurement of the actual carbon source concentration in Fed-Batch systems is difficult and is therefore estimated on the basis of the changes of measurable factors such as pH, dissolved oxygen, and the partial pressure of waste gases such as CO₂. Batch and Fed-Batch fermentations are common and well-known in the art and examples may be found in Brock (1989) Biotechnology: A Textbook of Industrial Microbiology, Second Edition, Sinauer Associates, Inc.

In some embodiments, continuous fermentation methods are used. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation allows for the modulation of one factor or any number of factors that affect cell growth or citrus terpenoid production. For example, one method maintains a limiting nutrient such as the carbon source or nitrogen level at a fixed rate and allows all other parameters to moderate. In other systems, a number of factors affecting growth can be altered continuously while the cell concentration (e.g., the concentration measured by media turbidity) is kept constant. Continuous systems strive to maintain steady state growth conditions. Thus, the cell loss due to media being drawn off is balanced against the cell growth rate in the fermentation. Methods of modulating nutrients and growth factors for continuous fermentation processes as well as techniques for maximizing the rate of product formation are well-known in the art of industrial microbiology and a variety of methods are detailed by Brock (1989) Biotechnology: A Textbook of Industrial Microbiology, Second Edition, Sinauer Associates, Inc.

In some embodiments, cells are immobilized on a substrate as whole cell catalysts and subjected to fermentation conditions for citrus terpenoid production.

In other embodiments, bottles of liquid culture are placed in shakers in order to introduce oxygen to the liquid and maintain the uniformity of the culture. In some embodiments, an incubator is used to control the temperature, humidity, shake speed, and/or other conditions in which a culture is grown. The simplest incubators are insulated boxes with an adjustable heater, typically going up to about 65° C. More elaborate incubators can also include the ability to lower the temperature (via refrigeration), or the ability to control humidity or CO₂ levels. Most incubators include a timer and some can also be programmed to cycle through different temperatures, humidity levels, etc. Incubators can vary in size from tabletop to units the size of small rooms.

If desired, a portion or all of the cell medium can be changed to replenish nutrients and/or avoid the build-up of potentially harmful metabolic byproducts and dead cells. In the case of suspension cultures, cells can be separated from the media by centrifuging or filtering the suspension culture and then resuspending the cells in fresh media. In the case of adherent cultures, the media can be removed directly by aspiration and replaced. In some embodiments, the cell medium allows at least a portion of the cells to divide for at least or about 5, 10, 20, 40, 50, 60, 65, or more cell divisions in a continuous culture (such as a continuous culture without dilution).

In certain embodiments, a recombinant host cell is cultured in a suitable medium (e.g., Luria-Bertoni broth, optionally supplemented with one or more additional agents, such as an inducer (e.g., where the terpene synthase and/or cytochrome P450-encoding nucleic acids are under the control of an inducible promoter); and the culture medium is overlaid with an organic solvent, e.g., dodecane, forming an organic layer. The citrus terpenoid produced by the recombinant host cell partitions into the organic layer, from which it can be purified. In accordance with this embodiment, the citrus terpenoid can be separated from other products which may be present in the organic layer. Separation of the citrus terpenoid from other products that may be present in the organic layer is readily achieved using, e.g., standard chromatographic techniques.

The citrus terpenoid can be further purified to obtain citrus terpenoid that is free from other isoprenoid compounds, macromolecules, contaminants, etc. In some embodiments, the citrus terpenoid is purified to e.g., at least about 40% pure, at least about 50% pure, at least about 60% pure, at least about 70% pure, at least about 80% pure, at least about 90% pure, at least about 95% pure, at least about 98%, or more than 98% pure.

Prior to subsequent to separation from other products and optionally purified, the citrus terpenoid can also be chemically modified in a cell-free reaction. By way of illustration, upon isolation of artemisinic acid from culture medium and/or a cell lysate, the artemisinic acid can be further chemically modified in a cell-free reaction to generate artemisinin.

### Example 1: Synthesis of Monoterpenes in Yeast

In S. cerevisiae GPP is synthesized via the mevalonate pathway by the condensation of isopentenyl pyrophosphate (IPP) and dimethylallyl pyrophosphate (DMAPP). To increase flux through the IPP/DMAPP pathway in the yeast host, several approaches are used: (i) integrate a truncated HMG-CoA reductase gene (tHMGR), which encodes a non-feedback-regulated rate-limiting enzyme of the mevalonate pathway (Ro, et al. (2006) Nature 440(7086):940-3); (ii) introduce a second copy of IDI1, the gene encoding IPP isomerase to increase DMAPP formation (Ignea, et al. (2011) Microb. Cell Fact 10:4); (iii) integrate a second copy of MAF1, a negative regulator of tRNA synthesis, to direct IPP away from tRNA synthesis and into GPP production (Liu, et al. (2013) J. Biotechnol. 168(4):446-451). Further, GPP production requires additional metabolic engineering because S. cerevisiae does not naturally produce GPP. The S. cerevisiae ERG20 gene encodes a farnesyl pyrophosphate synthase (FPPS) that, although having both GPP and FPP synthase activity, does not release GPP from its catalytic site (Fischer, et al. (2011) Biotechnol. Bioeng. 108(8):1883-92). Therefore, to synthesize free GPP, AgGPPS2, a GPP-specific synthase from grand fir (Abies grandis) is incorporated into this S. cerevisiae strain (Burke & Croteau (2002) Arch. Biochem. Biophys. 405(1):130-6). To further enhance GPP yields, the native ERG20 gene is replaced with mFPS144, a previously reported mutant FPPS (N144W mutation) from Gallus gallus, which has a much greater GPP synthase activity relative to FPP synthase activity (Stanley Fernandez, et al. (2000) Biochemistry 39(50):15316-21). The list of genes chromosomally integrated into the base yeast strain that produces GPP is provided in Table 2.

TABLE 2

| | GENBANK Gene | |
|---|---|---|
| Gene | ID | Reference |
| tHMGR¹ | 854900 | Ro, et al. (2006) Nature |
| | | 440(7086): 940-3 |
| IDI1 | 855986 | Ignea, et al. (2011) Microb. Cell |
| | | Fact 10:4 |
| MAF1 | 851568 | Liu, et al. (2013) J. Biotechnol. |
| | | 168(4): 446-451 |
| AgGPPS2 | AF513112³ | Burke & Croteau (2002) Arch. |
| | | Biochem. Biophys. 405(1): 130-6 |
| mFPS144² | 425061 | Stanley Fernandez, et al. (2000) |
| | | Biochemistry 39(50): 15316-21 |

¹531 amino acid residues of the N-terminus are removed.
²N144W mutation.
³GENBANK Accession No.

In combination with an appropriate yeast promoter (e.g., TDH3, ADH1 or TPI1) and terminator (e.g., ENO2, TPI1, ADH1 or PGK1), the open reading frame of a citrus monoterpene synthase is PCR-amplified and inserted into, e.g., a pXP vector (Fang, et al. (2011) Yeast 28(2):123-36) and integrated into the base yeast strain genome. Exemplary citrus monoterpene synthases are listed in Table 4.

TABLE 4

| | Accession | |
|---|---|---|
| Enzyme | No. | Reference |
| C. limon | AF514288 | Lucker, et al. (2002) Eur. J. |
| β-pinene | | Biochem. 269: 3160-71 |
| synthase | | |
| C. limon | AAM53943 | Lucker, et al. (2002) Eur. J. |
| γ-Terpinene | | Biochem. 269: 3160-71 |
| Synthase | | |
| C. limon | AAM53944 | Lucker, et al. (2002) Eur. J. |
| (R)-limonene | AAM53946 | Biochem. 269: 3160-71 |
| synthase | | |
| C. unshiu | BAD27257 | Shimada, et al. (2005) Sci. |
| D-limonene | | Hortic. 105: 507-12 |
| synthase | | |
| C. jambhiri | BAF73933 | Yamasaki & Akimitsu (2007) J. |
| Sabinene | | Plant Physiol. 164: 1436-48 |
| synthase | | |
| C. unshiu | BAP75561 | Shimada, et al. (2014) Plant |
| Linalool | BAP75560 | Sci. 229: 154-66 |
| synthase | BAP75559 | |
| C. jambhiri | BAM29049 | Shishido, et al. (2012) J. Plant |
| Geraniol | | Physiol. 169: 1401-7 |
| synthase | | |

The resulting yeast strain is cultured for a time sufficient for expression of the transgenes and accumulation of the citrus monoterpene (and optionally additional products). The supernatant and pellet of the cultures are separated by centrifugation. The pellet (intracellular material) is resuspended in solvent and lysed with glass beads. After centrifugation, the soluble portion is removed, evaporated to dryness, and resuspended to obtain the citrus terpenoid. Citrus terpenoids obtained by expression of the monoterpene synthases listed in Table include, but are not limited to, β-pinene, γ-terpinene, limonene, sabinene, linalool and geraniol.

### Example 2: Synthesis of Sesquiterpenes in Yeast

Yeast strains engineered to enhance carbon flux through the mevalonate pathway and accumulate high intracellular levels of farnesyl diphosphate (FPP) are used to facilitate sesquiterpene biosynthesis. Such strains include yeast strains SW24, CALI5-1, and CALI7-1 as described in U.S. Pat. No. 6,531,303, incorporated herein by reference.

The SW24 yeast strain was derived from wild-type strain ATCC 28383 (MATa) and was developed by mutagenesis of ATCC 28383 with nitrous acid followed by selection for growth in the presence of nystatin and exogenous cholesterol yielded a strain having an erg9 mutation (single base pair deletion) as well as an uncharacterized mutation supporting aerobic sterol uptake enhancement (sue). An additional round of chemical mutagenesis of the erg9 mutant with EMS and selection for 5-fluoroorotic acid resistant cells allowed for the isolation of a strain auxotrophic for uracil due to a mutation in the URA3 gene. This strain was genetically altered to contain a deletion in the HISS gene using a gene transplacement plasmid (Sikorski & Hieter (1989) Genetics 122:19-2) with the pop-in/pop-out gene replacement procedure (Rothstein (1991) Methods Enzymol. 194:281-30). The his3 mutant was named SWE23-ΔH1, which was further modified to contain mutations in the leu2 and trp1 genes using gene transplacement plasmids with the pop-in/pop-out gene replacement procedure as described above. One of the resulting strains containing erg9, ura3, his3, leu2, trp1, and sue mutations was further modified by exchanging the original erg9 frameshift mutation with the erg9Δ::HIS3 allele. The resulting strain is referred to as SW23B, and has the following genotype: ura3, leu2, trp1, his3, erg9::HIS3, sue. This strain was further modified by replacing the original ura3 mutation with a ura3Δ allele, resulting in strain SW24.

Citrus sesquiterpene synthase genes are engineered into a yeast expression vector (e.g., pESC-TRP or pESC-LEU from Strategene or Yep352-URA (Hill, et al. (1986) Yeast 2:163-167) and the recombinant vector is transformed into the SW24 yeast strain. An exemplary citrus sesquiterpene synthase is C. junos (E)-β-farnesene synthase known under Accession No. Q94JS8 (Maruyama, et al. (2001) Biol. Pharm. Bull. 24(10):1171-1175). The resulting yeast strain is cultured for a time sufficient for expression of the transgenes and accumulation of the citrus sesquiterpene (and optionally additional products). The supernatant and pellet of the cultures are separated by centrifugation. The pellet (intracellular material) is resuspended in solvent and lysed with glass beads. After centrifugation, the soluble portion is removed, evaporated to dryness, and resuspended to obtain the citrus terpenoid. Citrus terpenoids obtained by expression of the sesquiterpene synthases listed in Table 5 include, but are not limited to, trans-β-farnesene.

What is claimed is:

1. A method for producing a citrus terpenoid comprising
recombinantly expressing, in a host cell that produces isopentenyl pyrophosphate (IPP) and dimethylallyl pyrophosphate (DMAPP), one or more enzymes that convert the IPP and DMAPP to a citrus terpenoid; and
culturing the host cell to produce the citrus terpenoid.

2. The method of claim 1, wherein the IPP and DMAPP are produced by a mevalonate pathway, non-mevalonate pathway, or combination thereof.

3. The method of claim 1, wherein the one or more enzymes comprise terpene synthases, cytochrome P450s or a combination thereof.

4. A recombinant host cell that produces isopentenyl pyrophosphate (IPP) and dimethylallyl pyrophosphate (DMAPP) and expresses one or more enzymes that convert the IPP and DMAPP to a citrus terpenoid.

5. The recombinant host cell of claim 4, wherein the one or more enzymes comprise terpene synthases, cytochrome P450s or a combination thereof.


SEQUENCE LISTING (6 sequences)

SEQ ID NO: 1 - protein, 5 aa, Artificial sequence
  Synthetic peptide
  MISC_FEATURE (3)..(4): Xaa denotes any amino acid residue.
  MISC_FEATURE (5)..(5): Xaa denotes Asp or Glu.
DDXXX

SEQ ID NO: 2 - protein, 5 aa, Artificial sequence
  Synthetic peptide
  MISC_FEATURE (3)..(4): Xaa denotes any amino acid residue.
NDXXD

SEQ ID NO: 3 - protein, 11 aa, Artificial sequence
  Synthetic peptide
  MISC_FEATURE (1)..(1): Xaa denotes Leu or Val.
  MISC_FEATURE (2)..(2): Xaa denotes Leu, Val or Ala.
  MISC_FEATURE (3)..(3): Xaa denotes Asn or Asp.
  MISC_FEATURE (5)..(5): Xaa denotes Leu, Ile or Val.
  MISC_FEATURE (6)..(6): Xaa denotes any amino acid residue.
  MISC_FEATURE (7)..(7): Xaa denotes Ser or Thr.
  MISC_FEATURE (8)..(10): Xaa denotes any amino acid residue.
XXXDXXXXXXE

SEQ ID NO: 4 - protein, 11 aa, Artificial sequence
  Synthetic peptide
  MISC_FEATURE (3)..(10): Xaa denotes any amino acid residue.
RRXXXXXXXXW

SEQ ID NO: 5 - protein, 4 aa, Artificial sequence
  Synthetic peptide
  MISC_FEATURE (2)..(2): Xaa denotes any amino acid residue.
DXDD

SEQ ID NO: 6 - protein, 7 aa, Artificial sequence
  Synthetic peptide
  MISC_FEATURE (4)..(4): Xaa denotes any amino acid residue.
  MISC_FEATURE (7)..(7): Xaa denotes Ala or Gly.
GRRXCPX